(19) United States
(12) Patent Application Publication
(10) Pub. No.: US 2020/0054824 A1
(43) Pub. Date: Feb. 20, 2020

(54) A SYSTEM FOR THE HARVEST AND TRANSFER FOR HIGH VOLUME FAT GRAFTING USING A CENTRIFUGAL PUMP

(71) Applicant: Sarah Elizabeth HAGARTY, Rockford, IL (US)

(72) Inventor: Sarah Elizabeth HAGARTY, Rockford, IL (US)

(21) Appl. No.: 16/610,379

(22) PCT Filed: May 7, 2018
(86) PCT No.: PCT/US18/31433

§ 371 Date: Nov. 1, 2019

Related U.S. Application Data
(60) Provisional application No. 62/502,533, filed on May 5, 2017.

(51) Int. Cl. A61M 5/142; A61M 1/00; A61M 5/158

(52) U.S. Cl. CPC A61M 5/14236; A61M 1/0066; A61M 1/0056; A61M 1/0039; A61M 2210/0606; A61M 2202/08; A61M 2210/1007; A61M 2205/3334; A61M 2205/3365; A61M 5/158

(57) ABSTRACT

A closed loop system and methods for pumping harvested fat from a patient donor site to a patient grafting site using a centrifugal pump.

### CROSS REFERENCE TO RELATED APPLICATION

This application claims priority to U.S. Provisional Patent Application No. 62/502,533, titled “A CLOSED LOOP SYSTEM FOR DIRECT HARVEST AND TRANSFER FOR HIGH VOLUME FAT GRAFTING USING A CENTRIFUGAL PUMP,” filed May 5, 2017, incorporated herein by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This application related to the field of fat grafting and to embodiments of centrifugal pumping systems used for high volume fat grafting procedures.

### BACKGROUND

Fat grafting as an adjunct for contour modulation in breast reconstruction has become fairly commonplace¹⁻³ since The American Society of Plastic Surgeons lifted a ban on fat grafting to the breast in 2009. Optimal methods and techniques of transfer and fat handling has become an area of increased research and technological development. Many techniques involve the transfer of fat back into the breast via a hand held 60 cc syringe powered by manual plunger. Recently, Khouri^{4,5} has championed ‘megavolume fat transfer’ for breast reconstruction, designating mega as >300 cc per breast using this manual technique. Extrapolating this process to two breasts would necessitate at least ten 60 cc syringes. Transferring fat into syringes and refitting the plungers is tedious. Small amounts of fat may be spilt or lost with the filling of each syringe. Multiplying by ten syringes adds up to wasted viable tissue. Manually forcing the plunger after already performing manual or assisted liposuction is tiresome on the hands. What is needed are improved techniques to streamline this process, bypass the use of a syringe step, decrease operating time and anesthesia time for the patient.

### SUMMARY OF THE DISCLOSURE

In general, in one embodiment, a method of performing a closed loop fat harvesting and fat grafting process on a patient includes: (1) harvesting an amount of fat from a donor site of the patient; and (2) delivering a portion of the amount of harvested fat into a grafting site of the patient by adjusting the speed of a centrifugal pump to deliver the harvested fat at a rate of between 10 ml/min to 350 ml/min.

This and other embodiments can include one or more of the following features. The method can further include delivering a second portion of the amount of fat into a second grafting site of the patient at a rate of between 10 ml/min to 150 ml/min by adjusting the speed of the same centrifugal pump. The amount of fat from the harvesting step can be retained in a storage container and the delivering step can be performed by pumping the fat from the storage container directly to the grafting site using the centrifugal pump. In one aspect, the centrifugal pump is operated so that a portion of the amount of fat can be 700 cc and the delivering step is less than 22 minutes. The grafting site and the second grafting site can be on a breast. The grafting site can be on a right breast and the second grafting site can be on a left breast. Delivery of an amount of harvested fat to a grafting site with a centrifugal pump speed controlled to deliver an amount of harvested fat from about 1-5 ml, 1-50 ml, 1-100 ml, 100-200 ml, 100-500 ml, 100-1000 ml or 100-2000 ml wherein the harvested fat is delivered by operation of the centrifugal pump connected directly to the fat harvesting container. In one aspect, the speed of the centrifugal pump may be selected to deliver harvested fat at a rate for a fat grafting process performed as part of a breast reconstruction procedure or a cosmetic breast revision procedure. In one aspect, the speed of the centrifugal pump may be selected to deliver harvested fat at a rate for a fat grafting process performed as part of a cosmetic body contouring procedure. In one aspect, the speed of the centrifugal pump may be selected to deliver harvested fat at a rate adapted as a result of a portion of the harvesting step or the delivering step adjusted to accommodate for a partial defect at the grafting site. The method can further include adapting a portion of the harvesting step or the centrifugal pump operation during the delivering step to accommodate for a scarring from radiated tissue at the grafting site. The method can further include adapting a portion of the harvesting step or the delivering step to accommodate for an implant volume inadequacy at the grafting site.

In general, in one embodiment, a method of performing a fat grafting process on a patient includes: (1) harvesting an amount of fat from a donor site of the patient into a storage container; (2) treatment or preparation of the fat in various ways (for example centrifuging, filtering, gravitational separation, using the Revolve® system) (3) positioning a delivery cannula into a first portion of a grafting site of the patient; (4) pumping a portion of the amount of fat directly from the storage container via a centrifugal pump into the first portion of the grafting site of the patient at a rate of between 100 ml/min to 350 ml/min until a first volume of fat is indicated on a pump flow meter or other measure used during the pumping step; and (5) positioning a delivery cannula into a second portion of a grafting site of the patient; and (6) pumping a second portion of the amount of fat directly from the storage container via the same centrifugal pump into the second portion of the grafting site of the patient at a rate of between 10 ml/min to 350 ml/min until a second volume of fat is indicated on a pump flow meter or other measure used during the pumping step, wherein the centrifugal pump used during the pumping step is in continuous communication with the storage container during the positioning a delivery cannula steps. In some embodiments, the harvested fat and pump operations are provided by a pump-fat characterization table.

In general, in one embodiment, a method of performing a fat grafting process on a patient includes: (1) harvesting an amount of fat from a donor site of the patient into a collection canister; (2) treatment or preparation of the fat in various ways (for example centrifuging, filtering, gravitational separation, using the Revolve® system), (3) positioning a delivery cannula into a first portion of a grafting site of the patient; (4) operating a centrifugal pump for pumping a portion of the amount of fat directly from the collection canister into the first portion of the grafting site of the patient at a rate of between 10 ml/min to 350 ml/min until a first volume of fat is indicated by the pump used during the pumping step; and (5) positioning the delivery cannula into a second portion of a grafting site of the patient; and (6) operating a centrifugal pump for pumping a second portion of the amount of fat directly from the collection canister into the second portion of the grafting site of the patient at a rate of between 10 ml/min to 350 ml/min until a second volume of fat is indicated by the pump used during the pumping step, wherein a single piece of tubing less than 12 inches long connects the harvesting canister to the centrifugal pump inlet and a single piece of tubing less than 36 inches long connects the centrifugal pump outlet to the delivery cannula.

These and other embodiments can include one or more of the following features. The method can further include operating a centrifugal pump used in the pumping a portion steps continuously while performing the positioning the delivery cannula steps. The method can further include operating the pump used in the pumping a portion step at a first pump speed for a first flow rate during the pumping a portion step and at a second pump speed for a second different flow rate during the pumping a second portion step.

In general, in one embodiment, a method of performing a fat grafting process on a patient includes: (1) pre-treating and then harvesting an amount of fat from a donor site of the patient into a storage container; (2) treatment or preparation of the fat in various ways (for example centrifuging, filtering, gravitational separation, to provide harvested fat for a delivery using a centrifugal pump as characterized as in FIG. 45; ((3) positioning a delivery cannula into a first port of a grafting site of the patient; (4) setting the speed of pump rotation for pumping a portion of the amount of fat directly from the storage container into the first port of the grafting site of the patient until a first volume of fat is pumped; and (5) positioning the delivery cannula into a second port of a grafting site of the patient; and (6) setting the speed of pump rotation for pumping a second portion of the amount of fat directly from the storage container into the second port of the grafting site of the patient until a second volume of fat is pumped, wherein the pump used during the pumping step is in continuous communication with the storage container during the positioning a delivery cannula steps.

These and other embodiments of the closed loop centrifugal pump fat pumping method can include one or more of the following features. The speed of the pump is adjusted for pumping a portion of the fat at a rate of 10 ml/min to 350 ml/min for delivery to a grafting site. The grafting site of the patient can include a portion of the patient's face. The grafting site of the patient can include a portion of the patient's buttocks. The grafting site of the patient can include a portion of the patient's breast. The steps can be performed to accomplish a cosmetic effect at the grafting site. The steps can be performed to accomplish a reconstructive effect at the grafting site. The steps can be performed to accomplish a structural revision effect at the grafting site or any other part of the body with a soft tissue defect of either reconstructive or cosmetic nature, including by way of example, a scar deformity or other contour abnormality anywhere on the body. The steps can be performed to accomplish a bilateral breast construction at the grafting site. The steps can be performed as part of a cosmetic body contouring procedure.

In general, in one embodiment, a method of performing a fat grafting process on a patient includes (1) harvesting an amount of fat from a donor site of the patient; and (2) operating a centrifugal pump for delivering a portion of the amount of fat into a grafting site of the patient at a rate of between 100 ml/min to 150 ml/min.

This and other embodiments can include one or more of the following features. The method can further include operating the centrifugal pump for delivering a second portion of the amount of fat into a second grafting site of the patient at a rate of between 100 ml/min to 150 ml/min. The amount of fat from the harvesting step can be retained in a storage container and the delivering step can be performed by pumping the fat from the storage container directly to the grafting site. The fat from the harvesting step and delivered by operating a centrifugal pump can remain within the sterile field established for performing the fat grafting process. The portion of the amount of fat can be 700 cc and the delivering step can be less than 22 minutes. The grafting site and the second grafting site can be on a breast. The grafting site can be on a right breast and the second grafting site can be on a left breast. The harvesting an amount can be between about 1-5 ml, 1-50 ml, 1-100 ml, 100-200 ml, 100-500 ml, 100-1000 ml or 100-2000 ml of harvested fat. Fat grafting process can be performed as part of a breast reconstruction procedure or a cosmetic breast revision procedure. Fat grafting process can be performed as part of a cosmetic body contouring procedure. The method can further include adapting a portion of the harvesting step or the operating step to accommodate for a partial defect at the grafting site. The method can further include adapting a portion of the harvesting step or the operating step to accommodate for a scarring from radiated tissue at the grafting site. The method can further include adapting a portion of the harvesting step or the operating step to accommodate for an implant volume inadequacy at the grafting site. The operating step can be performed by imparting rotation of the centrifugal pump via a magnetic coupling. The operating step can be performed by imparting rotation of the centrifugal pump using a surgical drill, or a motor drive or a flexible cable drive. The amount of fat delivered can be measured by a component of the centrifugal pump or a flow meter coupled to a centrifugal pump outlet or by operating the centrifugal pump at a predetermined speed setting for a predetermined time duration.

In general, in one embodiment, a method of performing a fat grafting process on a patient includes: (1) harvesting an amount of fat from a donor site of the patient into a storage container; (2) positioning a delivery cannula into a first portion of a grafting site of the patient; (3) adjusting the speed of a centrifugal pump to pump a portion of the amount of fat directly from the storage container into the first portion of the grafting site of the patient at a rate of between 10 ml/min to 350 ml/min until a first volume of fat is delivered; (4) positioning a delivery cannula into a second portion of a grafting site of the patient; and (5) adjusting the speed of the centrifugal pump to pump a second portion of the amount of fat directly from the storage container into the second portion of the grafting site of the patient at a rate of between 10 ml/min to 350 ml/min until a second volume of fat is delivered, wherein the centrifugal pump remains within the sterile field and in fluid communication with the storage container.

In general, in one embodiment, a method of performing a fat grafting process on a patient includes: (1) harvesting an amount of fat from a donor site of the patient into a collection canister; positioning a delivery cannula into a first portion of a grafting site of the patient; (2) flowing a portion of the amount of fat directly from the collection canister through a first tube and into an inlet of a centrifugal pump; (3) adjusting the speed of the centrifugal pump to deliver fat from a collection canister through a pump outlet into a second tubing connected to the delivery cannula into the first portion of the grafting site of the patient at a rate of between 10 ml/min to 350 ml/min until a first volume of fat is delivered; (4) positioning the delivery cannula into a second portion of a grafting site of the patient; and (5) flowing a second portion of the amount of fat directly from the collection canister through the delivery cannula into the second portion of the grafting site of the patient by adjusting the speed of the centrifugal pump to deliver fat at a rate of between 10 ml/min to 350 ml/min until a second volume of fat is delivered, wherein the first tube and the second tube remain completely within the sterile field and the combined length of the first tube and the second tube is less than four feet.

These and other embodiments can include one or more of the following features. The method can further include operating the centrifugal pump used in the flowing a portion steps continuously while performing the positioning the delivery cannula steps. The method can further include operating the centrifugal pump used in the flowing a portion step at a first flow rate during the pumping a portion step and at a second different flow rate during the flowing a second portion step.

In general, in one embodiment, a method of performing a fat grafting process on a patient includes: (1) harvesting an amount of fat from a donor site of the patient into a storage container; positioning a delivery cannula into a first port of a grafting site of the patient; (2) operating a centrifugal pump at a first speed to pump a portion of the amount of fat directly from the storage container into the first port of the grafting site of the patient until a first volume of fat is delivered; (3) positioning the delivery cannula into a second port of a grafting site of the patient; and (4) operating the centrifugal pump at a second speed to pump a second portion of the amount of fat directly from the storage container into the second port of the grafting site of the patient until a second volume of fat is delivered, wherein the centrifugal pump used to pump the fat is at least partially underneath or directly adjacent to the storage container such that a tube connecting a storage container outlet to a pump inlet is less than 12 inches long.

This and other embodiments can include one or more of the following features. One or both of the steps of operating a centrifugal pump can be performed at a rate of 100 ml/min to 350 ml/min. The grafting site of the patient can include a portion of the patient's face. The grafting site of the patient can include a portion of the patient's buttocks. The grafting site of the patient can include a portion of the patient's breast. The steps can be performed to accomplish a cosmetic effect at the grafting site. The steps can be performed to accomplish a reconstructive effect at the grafting site. The steps can be performed to accomplish a structural revision effect at the grafting site. The steps can be performed to accomplish a bilateral breast construction at the grafting site. The steps can be performed as part of a cosmetic body contouring procedure. The methods can further include manipulating or treating at least a portion of the amount of fat from a harvesting step. A step of manipulating or treating can include one or more of a step of centrifuging harvested fat: (1) a step of processing fat to meet a characterization property of FIG. 45; (2) a step of filtering harvested fat; (3) a step of gravitational separation of harvested fat; (4) or a step of mixing harvested fat; (5) or a step of incorporating one or more substances into harvested fat helpful to ensuring viability of fat cells for the pumping process or for survival after a grafting process. The method can further include positioning the collection canister and the centrifugal pump in a position adjacent the patient the patient's fat may be harvested and the fat delivered using the centrifugal pump without using any tubing longer than three feet.

In general, in one embodiment, a method of performing a closed loop fat harvesting and fat grafting process on a patient includes: (1) harvesting an amount of fat from a donor site of the patient; and (2) delivering a portion of the amount of harvested fat into a grafting site of the patient by adjusting the speed of a centrifugal pump to deliver the harvested fat at a rate of between 10 ml/min to 350 ml/min.

This and other embodiments can include one or more of the following features. The method can further include delivering a second portion of the amount of fat into a second grafting site of the patient at a rate of between 10 ml/min to 150 ml/min by adjusting the speed of the same centrifugal pump. The amount of fat from the harvesting step can be retained in a storage container and the delivering step can be performed by pumping the fat from the storage container directly to the grafting site using the centrifugal pump. The centrifugal pump can be operated so that a portion of the amount of fat is 700 cc and the delivering step is less than 22 minutes. The grafting site and the second grafting site can be on a breast. The grafting site can be on a right breast and the second grafting site can be on a left breast. The delivery of an amount of harvested fat to a grafting site with a centrifugal pump speed controlled to deliver an amount of harvested fat can be from about 1-5 ml, 1-50 ml, 1-100 ml, 100-200 ml, 100-500 ml, 100-1000 ml or 100-2000 ml. The harvested fat can be delivered by operation of the centrifugal pump connected directly to the fat harvesting container. The speed of the centrifugal pump can be selected to deliver harvested fat at a rate for a fat grafting process performed as part of a breast reconstruction procedure or a cosmetic breast revision procedure, or for a fat grafting process performed as part of a cosmetic body contouring procedure, or at a rate adapted as a result of a portion of the harvesting step or the delivering step adjusted to accommodate for a partial defect at the grafting site or for adapting a portion of the harvesting step or the centrifugal pump operation during the delivering step to accommodate for a scarring from radiated tissue at the grafting site or for adapting a portion of the harvesting step or the delivering step to accommodate for an implant volume inadequacy at the grafting site. The method can further include one or more or a combination of the steps of performing a fat grafting process on a patient including: (1) harvesting an amount of fat from a donor site of the patient into a storage container; (2) treatment or preparation of the fat by centrifuging, filtering, or gravitational separation; (3) positioning a delivery cannula into a first portion of a grafting site of the patient; (4) pumping a portion of the amount of fat directly from the storage container via a centrifugal pump into the first portion of the grafting site of the patient at a rate of between 100 ml/min to 350 ml/min until a first volume of fat is indicated on a pump flow meter or other measure used during the pumping step; and (5) positioning a delivery cannula into a second portion of a grafting site of the patient; and (6) pumping a second portion of the amount of fat directly from the storage container via the same centrifugal pump into the second portion of the grafting site of the patient at a rate of between 10 ml/min to 350 ml/min until a second volume of fat is indicated on a pump flow meter or other measure used during the pumping step, wherein the centrifugal pump used during the pumping step is in continuous communication with the storage container during the positioning a delivery cannula steps.

In general, in one embodiment, a system for closed loop fat harvesting and continuous fat pumping includes a centrifugal pump having an inlet, an outlet and a drive shaft, a first length of tubing for connecting an outlet of a collection canister to the inlet, a second length of tubing for connecting the outlet to a delivery cannula, and a drive system coupled to the drive shaft wherein operation of the drive system rotates the drive shaft to pump fat from the collection canister to the delivery cannula at a rate of fat delivery.

This and other embodiments can include one or more of the following features. The first length of tubing can be selected to extend from the outlet of the collection canister to the inlet when the centrifugal pump is at least partially underneath or adjacent or in proximity to the collection canister. The delivery cannula can be a 12 gauge microinjection cannula, a 14 gauge microinjection cannula or a 16 gauge microinjection cannula. A fat harvesting cannula used to fill the collection canister can be a 2 mm size cannula, a 3 mm size cannula, a 4 mm size cannula or a 5 mm size cannula. The first length of tubing can be less than 12 inches long. The second length of tubing can be less than 48 inches long. The centrifugal pump can have operating characteristics in the fat delivery mode adapted to perform according to a pump characterization table as in FIG. 45. The centrifugal pump can have an impeller without vanes, an impeller with vanes or an impeller shaped as a spiral drive, a helical drive, a worm drive of a section of any of the above. The storage canister can have a size to accommodate the volumes. The operation of the drive system can be adapted and configured to rotate the drive shaft of the centrifugal pump to provide fat as part of any surgical procedure or to provide fat pumping rates as provided in a pump-fat characterization table as in FIG. 45. The drive shaft can be any of a surgical drill, a motor drive, a magnetic drive or a flexible couple drive.

In general, in one embodiment, a pump includes a housing with an interior chamber, an inlet in communication with the interior chamber, an outlet in communication with the interior chamber, and an impeller within the interior chamber and shaft coupled to the impeller wherein rotation of the impeller shaft causes a volume of harvested fat at the inlet to move through the interior chamber with the impeller and exit of the interior chamber via the outlet.

This and other embodiments can include one or more of the following features. The impeller can be rotated by a magnetic drive coupled to the housing. The impeller shaft can extend outside of the pump housing via a aperture in the housing. The impeller can be rotated by coupling a portion of the impeller shaft outside of the pump housing to a drill. The impeller can be rotated by coupling a portion of the impeller shaft outside of the pump housing to a motor. The impeller can be rotated by coupling a portion of the impeller shaft to a flexible driver system. The impeller shaft can be a flexible cable. The impeller shaft can have a fitting to receive a drive shaft via an aperture in the housing adjacent to the fitting. The fitting and the drive shaft can be keyed to have a complimentary, releaseable engagement. The releaseable engagement can be male/female, socket/driver, start shaped, indexed or other complementary engagement pattern adapted and configured for coupling the fitting and the drive shaft. A speed of rotation of the impeller can provide a fat flow rate of between 10-50 ml/min. A speed of rotation of the impeller can provide a fat flow rate of between 75-200 ml/min. A speed of rotation of the impeller can provide a fat flow rate of between 200-400 ml/min. A first speed of impeller rotation can provide a user determined low volume fat pumping rate. A second speed of impeller rotation can provide a user determines medium volume fat pumping rate. A third speed of impeller rotation can provide a user determined high volume fat pumping rate. The pump can further include a drive system for controlling impeller rotation speed wherein the drive system if adapted and configured to provide adjustable impeller speed or preselected impeller speed within the range of 10 ml/min to 400 ml/min. The pump, the fat and the impeller rotation speed can be characterized and provided as in FIG. 45. The impeller can have a smooth pumping surface. The impeller can have one or more raised surfaces in a pumping surface. The impeller can be a spiral drive, a helix drive or a worm gear drive or a section of any of these drives.

### BRIEF DESCRIPTION OF THE DRAWINGS

FIG. 1 is a top down view of the separate components for a closed loop system and how they are connected together with respect to the flow and direction of the fat harvesting from the patient to a centrifugal pump for delivery back to the patient. This view includes a lipo-aspirate harvesting cannula (large at bottom) connected directly to the fat harvesting canister. There is also tubing connecting the collection canister to the centrifugal pump inlet and tubing connecting the centrifugal pump outlet to the fat introduction cannula.

FIG. 2 is a perspective view of an embodiment of a centrifugal fat transfer pump.

FIG. 3 is a perspective view of another embodiment of a centrifugal fat transfer pump.

FIG. 4 is a front view of an integrated infusion pump and centrifugal pump for use in closed loop fat grafting.

FIG. 5 is a flow chart of an exemplary embodiment of a centrifugal pump closed loop high volume fat transfer method.

FIG. 6 is a front view of an exemplary the control panel, display, foot pedal and cable for a centrifugal pump configured for fat transfer.

FIGS. 7A and 7B illustrate pre-cut tubing lengths and front views, respectively, of tubing within packaging and, optionally, with fittings for use with components of the closed loop centrifugal fat pumping system.

FIG. 8 is a perspective view of a fat connection canister after or during a fat harvesting step and shown connected to an end of fat transfer tubing for connection to an inlet of a centrifugal pump described herein.

FIGS. 9A, 9B and 9C are side and top view of a mesh filter, internal propeller and top connections, respectively, of an alternative fat harvesting canister.

FIGS. 10A and 10B are top views of the distal ends of cannulas suited to the fat delivery method.

FIGS. 11A and 11B are top view of the proximal ends of cannulas suite to the fat delivery method.

FIG. 12A is a perspective view of a second end of the second tubing (TL2) connected to a delivery cannula inlet (Ci).

FIG. 12B is a perspective view of the first tubing (TL1 not connected) and the second tubing, with the second end connected to a delivery cannula inlet (Ci).

FIG. 13 is a top view of a pump according to the present invention.

FIG. 14 is a sectional view of the pump of FIG. 13 taken along line 2-2.

FIG. 15 is a perspective view of the lower section and separating surfaces of the impeller (i.e., vanes) of FIGS. 13 and 14.

FIGS. 16 and 17 are perspective views of alternative impeller configurations having separating surfaces of various sizes and orientations.

FIG. 18 is a partial view indicating a preferred form of pump having a built-in drive motor for the accelerator or rotator of the pump.

FIG. 19 is an axial cross-sectional view of another form of pump having a built-in drive motor for the accelerator or rotator assembly of the pump.

FIG. 20 is a vertical cross-sectional view taken at line 3-3 of FIG. 19.

FIG. 21 is an axial cross-sectional view showing still another form of pump according to the invention.

FIG. 22 is a partial cross-sectional view taken at line 5-5 of FIG. 21.

FIG. 23 is an axial cross-sectional view showing another form of pump according to the invention.

FIG. 24 is a perspective view showing a modified form of accelerator or rotator useful in pumps according to the invention.

FIG. 25 is an elevational view of the apparatus shown in FIG. 24, taken from the right hand of the apparatus of FIG. 24.

FIG. 26 shows another modified form of accelerator or rotator.

FIGS. 27-32 show six additional forms of accelerators or rotators useful in the pumps of FIGS. 18-26 according to various other embodiments.

FIG. 33 is a partial sectional view of the pump of the invention;

FIG. 34 is a sectional view of the pump of the invention taken along line 4-4 of FIG. 33;

FIG. 35 is a sectional view of the pump of the invention taken along line 5-5 of FIG. 33;

FIG. 36 is an illustrative view of the helical-shaped foil impeller of the invention depicted in a two dimensional plane; and

FIG. 37 is a sectional view of the pump of the invention taken along lines 7-7 of FIG. 33.

FIGS. 38A and 38B illustrate a pump casing with female socket, a base and a drill apart (FIG. 38A) and then assembled for operation to pump fat using the drill to drive the pump components (FIG. 38B).

FIG. 38B is components of FIG. 38A assembled for use to perform a fat transfer procedure as described herein.

FIG. 39 is a view of a centrifugal pump embodiment with a shaft extending from the pump housing and engaged with a variable speed surgical drill having a female socket receiver.

FIG. 40 is a top down view of a stabilizing plate used as a base for a centrifugal pump coupled to a surgical drill.

FIG. 41A is an interior view of a centrifugal pump with the cover removed to show the impeller and vanes. FIG. 41B is a rear view of the centrifugal pump in FIG. 41A. The drive shaft of the pump extends from the casing.

FIG. 42 is a perspective view of one arrangement of a centrifugal pump coupled to a surgical drill and configured for pumping the contents of the fat in a harvesting canister to the delivery tubing and to a delivery cannula. The centrifugal pump and surgical drill are mounted onto a plate and positioned on the tabletop within the sterile field. One or both of the centrifugal pump and the surgical drill or a portion of each of the centrifugal pump and the surgical drill are beneath the fat harvesting canister or within the footprint of the canister stand.

FIG. 43 is a perspective view of one arrangement of a centrifugal pump coupled to a drive shaft and configured for pumping the contents of the fat in a harvesting canister to the delivery tubing and to a delivery cannula. The centrifugal pump and drive shaft are mounted onto a plate and positioned on the tabletop within the sterile field. One or both of the centrifugal pump and the drive shaft or a portion of each of the centrifugal pump and the drive shaft are beneath the fat harvesting canister or within the footprint of the canister stand. A controller for adjusting the speed of the drive shaft and the pumping speed of the centrifugal pump is located outside of the sterile field. A foot pedal coupled to the drive shaft speed controller is located within the surgical field.

FIG. 44 is a perspective view of one arrangement of a centrifugal pump coupled to a drive shaft and configured for pumping the contents of the fat in a harvesting canister to the delivery tubing and to a delivery cannula. The centrifugal pump and drive shaft are mounted onto a plate and positioned on the tabletop within the sterile field. One or both of the centrifugal pump and the drive shaft or a portion of each of the centrifugal pump and the drive shaft are beneath the fat harvesting canister or within the footprint of the canister stand. A controller (not shown) for adjusting the speed of the drive shaft and the pumping speed of the centrifugal pump is located outside of the sterile field. A foot pedal coupled to the drive shaft speed controller is located within the surgical field.

FIG. 45 is a table of exemplary centrifugal pump characterization chart for centrifugal pump operations in conjunction with existing surgical drills. The table relates the speed (rpm) setting for the drill model for an estimated flow rate of delivered fat. The table also includes a range of fluid percentages (FL %) for the harvested fat and for correlating the various fluid percentages to the flow rates. The table also includes representative High (H), Medium (M) and Low (L) fat flow rates. Characterization studies of fat composition and fluid amounts may be performed and recorded in the chart portions (a) and (b). A user may select different H, M and L flow rates. Drill manufacturers may provide this information as an operational suggestion as the surgical drill manufacturer tests or certifies a particular drill for use with a centrifugal fat pump.

FIG. 46 is a side view of one arrangement of first location (L1) and a second location (L2) for positioning a centrifugal pump and a drive unit on a tray or table. The first and second locations are selected to place all or a portion of one or both of the centrifugal pump and drive unit under the harvesting canister or within the foot print of the canister frame. The harvesting frame may be modified to increase the height of the bottom of the canister from the tope of the tray for this purpose.

### DETAILED DESCRIPTION

In one aspect, there is provided method of harvesting and transferring large volumes of fat for grafting in a sterile closed circuit system. Embodiments of the inventive centrifugal pump enabled fat delivery system and methods provide a number of benefits, including:

a) Saves operating and anesthesia times

b) Limits spillage and waste of graft tissue

c) Decreases manual force needed

d) Decreases strain and fatigue on the surgeon's hands, and

e) Limits air exposure of graft material.

f) Maintains harvested fat storage, tubing and pump in the sterile field throughout entire process.

In one specific aspect, the same size, gauge and type of tubing used during a tumescent infusion step is used for connections to a centrifugal pump for delivery of harvested fat. More specifically, an infusion cannula and infusion tubing is coupled to an infusion pump and used for infiltration of fluids at the fat harvest site. The fat is harvested into a fat collection container. The fat may be processed according to a surgeon's individual preference. As in for example centrifuging, filtering, gravitational separation, using the Revolve® system. The Revolve® system may be used in the closed loop circuit as the collection canister.

Optionally, the procedures used during tumescent infusion and/or processing of harvested fat may be standardized, controlled or measured to provide better pump delivery certainty (see FIG. 45). Still further, increased understanding of the characteristics of harvested and processed fat will aid users in approximating procedure times and improve understanding of selection from among the various difference centrifugal pump designs for the different circumstances and clinical needs for fat pumping.

Thereafter, a first section or length of tubing is connected between the collection container outlet and a centrifugal pump inlet and another section or length of tubing connects the centrifugal pump outlet to a suitable delivery cannula. Thereafter, the centrifugal pump is operated to provide controllable rate, fat delivery to the fat grafting site via a suitable delivery cannula.

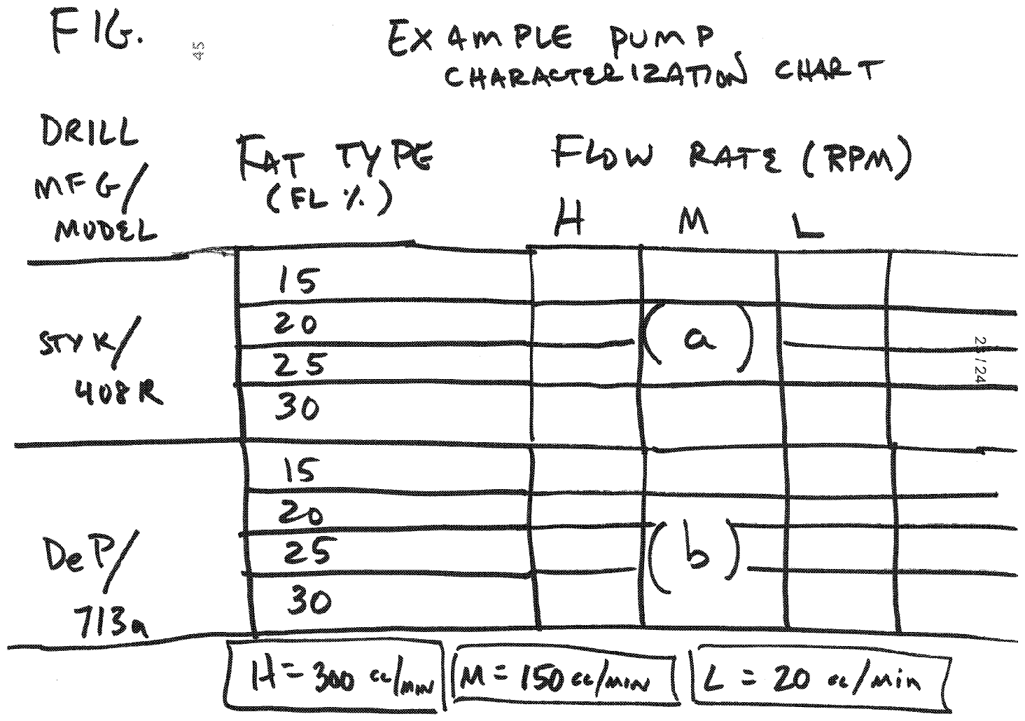

In various alternative aspects of this invention elaborate on the construction of a simple closed loop system for continuous flow harvesting, collection, syphoning, and grafting of large volumes of fat. As a result, this inventive system of methods uniquely bypass standard cumbersome and time-consuming process, mainly the need to first transfer fat into individual syringes prior to grafting into the patient or the use of pumping systems outside of the sterile field and the resulting excessive tubing lengths involved therewith. Previous methods are traditionally done with unmeasured, manual pressures as generated by a simple plunger syringe. It can be messy and sloppy, with loss of fat. In contrast, this innovative methods and system presented here simplifies the whole process into a closed loop, from the patient donor harvest site, and then back to the patient. The major benefit is a significant reduction in time to transfer fat from patient donor or site to patient graft site. Additional advantages include a simplification of the process with less harvested fat air exposure time, consistent controlled limit on flow pressures, and a simple centrifugal pump speed control that can be operated by an assistant, held in one hand, or controlled by foot pedal. Still further, the system is adaptable to standard liposuction and introduction cannulas through standard liposuction tubing and a luer lock connection. Moreover, the collection canisters are also interchangeable. Advantageously, the frame or stand supporting the canister may be easily modified to provide vertical clearance for placement of one, both or a portion of each of the centrifugal pump, drive unit, drill or flexible cable into an orderly and compact footprint. (See FIGS. 42, 43, 44 and 46). In contrast to the prior fat harvesting systems, embodiments of the closed loop pumping system described herein maintain the fat delivery pump and all the delivery tubing within the sterile field in an organized arrangement within easy reach of the user (FIG. 46).

While desiring not to be bound by theory, it is believed that a number of the design factors used for centrifugal blood pumps may be applied to the various designs herein for high volume centrifugal pumping of harvested fat. Like blood, harvested fat is a complex and delicate fluid. The constituents of suctioned and harvested fat are fairly readily affected by the manner in which fat is physically handled or treated. Fat subjected to mechanical shear, to impact, to depressurization, or the like, may be damaged, the balance between the constituents may be affected, or deterioration may result from physical mishandling. Fat which has been damaged may be unfit for use, provide inferior results or lead to complications during recovery.

Various other centrifugal pump configurations, dimensions and designs are possible to provide controllable delivery of high volume fat transfers. In the various embodiments described herein, the pump designs shown and described have in common that the harvested fat is handled gently, meaning that shear, shock, vibration, impact, severe pressure or temperature change, or any other condition or treatment which would unduly damage the fat is mitigated or maintained within an operating range wherein the harvested fat remains viable for the intended surgical or clinical benefit as described herein. Put another way, the centrifugal pump designs described herein may be modified and operated whereby the pumped fat is exposed to nonturbulent flow or limited turbulent flow with a corresponding gradual and smooth acceleration. In other words, the centrifugal pumps for fat transfer are configured to transfer fat at the desired rates while maintaining an operating environment that does no, little or only recoverable hard to the pumped fat.

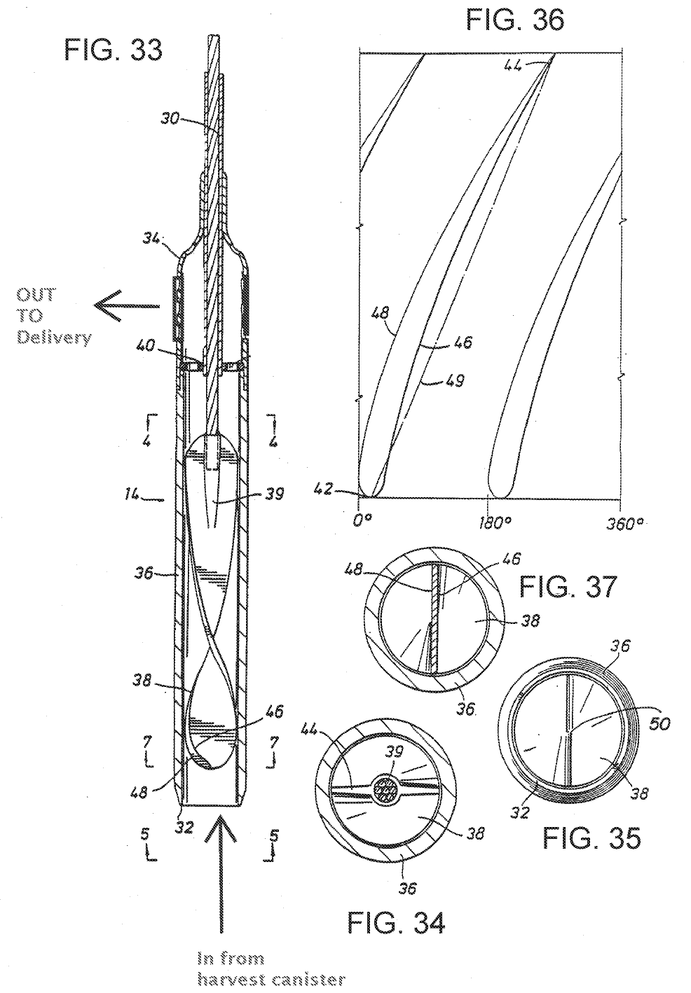

In various alternative embodiments, the forms of the pump housings, rotors, vanes, impellers or rotators may vary considerably. For example, the rotating structures that will engage to pump the fat may be constructed entirely or partly of porous or perforate materials, i.e., the vanes of the rotator which accelerate the fluid circularly may be made of screen, of perforate plates or sheets, of spaced rods, or the like, and will still ably perform their fluid accelerating function while providing damage free or limited harm controlled rate pumping of harvested fat. Additionally, rotators, vanes, impellers, dividers or other pump structures may be of axially extended form, so that the fluid is accelerated axially or axially and radially. Designs of this nature would extend the flow path from inlet to outlet so that acceleration would be at a slower rate. In some of the centrifugal pump embodiments shown and/or described herein, one or more tangential outlets could be provided, disposed in the direction of fluid flow inside the peripheral wall of the pump. In multistage pumps, the several rotators, impellers, blades, vanes which may be alike or unlike, may be driven at different rotational speeds. The axes of multistage rotators may be offset and in other positions out of alignment. In other embodiments, the impellers or rotating pumping surfaces may be arranged to provide a fat flow axially from the inlet to the outlet with respect to the pump casing as in FIGS. 33, 40, 41A, 41B and 44.

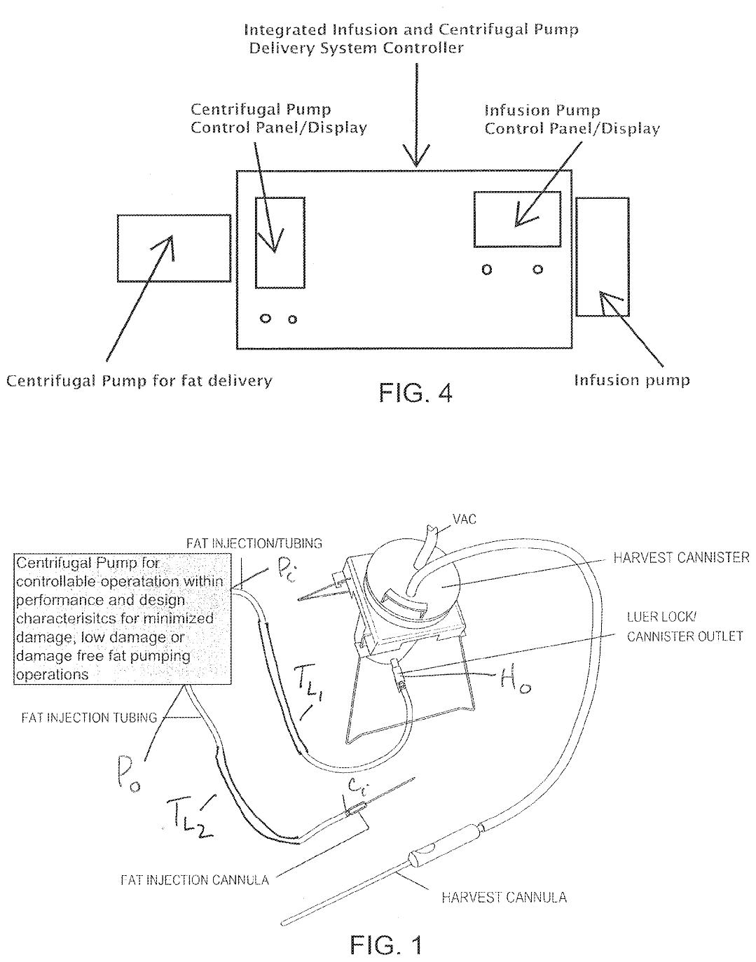

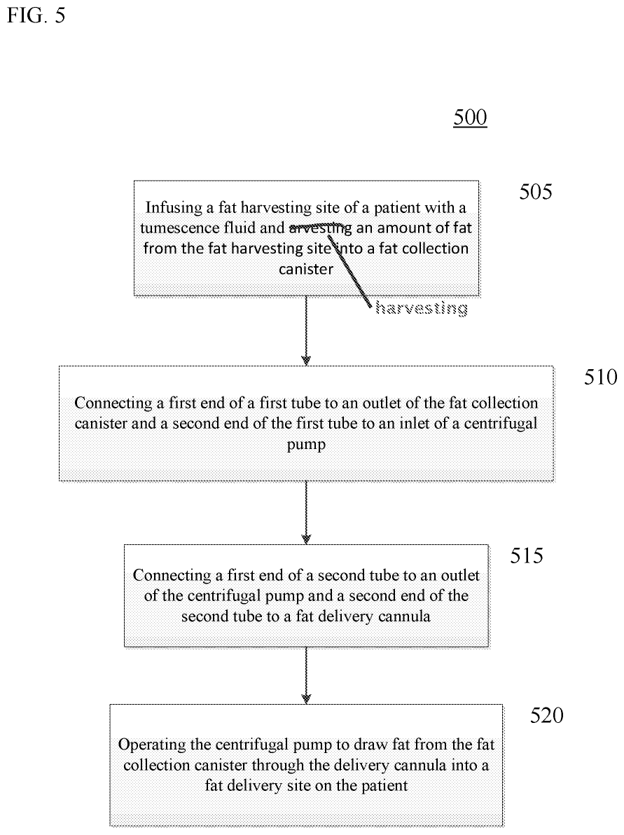

A method of providing high volume fat grafting may be appreciated by reference to FIGS. 1 and 5. FIG. 1 shows a schematic view of an exemplary closed circuit fat transfer system. FIG. 1 shows how the harvesting cannula, collection canister, centrifugal pump and delivery cannula are connected as one system—harvesting, processing and transfer with minimal air exposure and short time out side of the patient. The representative box in FIG. 1 could be any of the various centrifugal pump embodiments described herein.

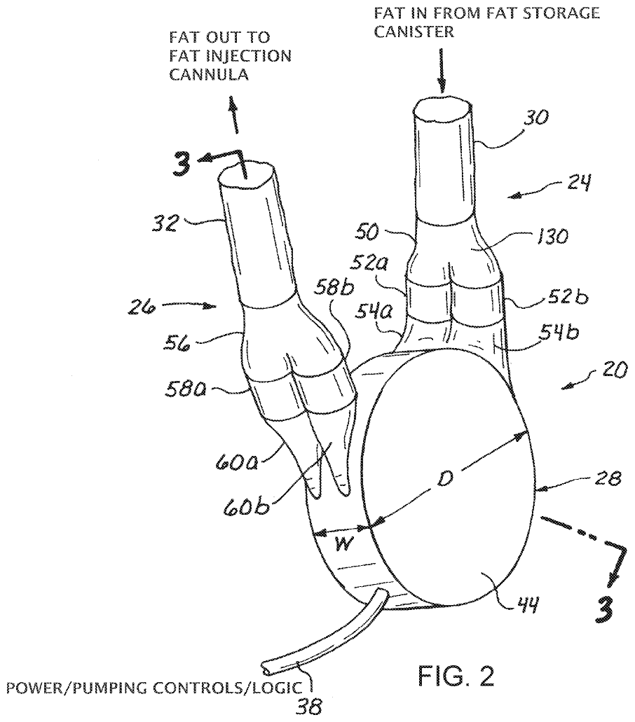

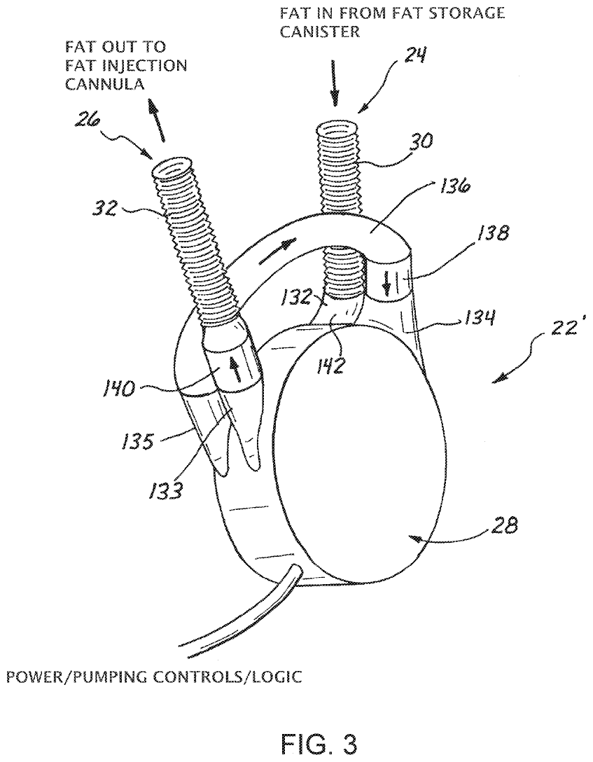

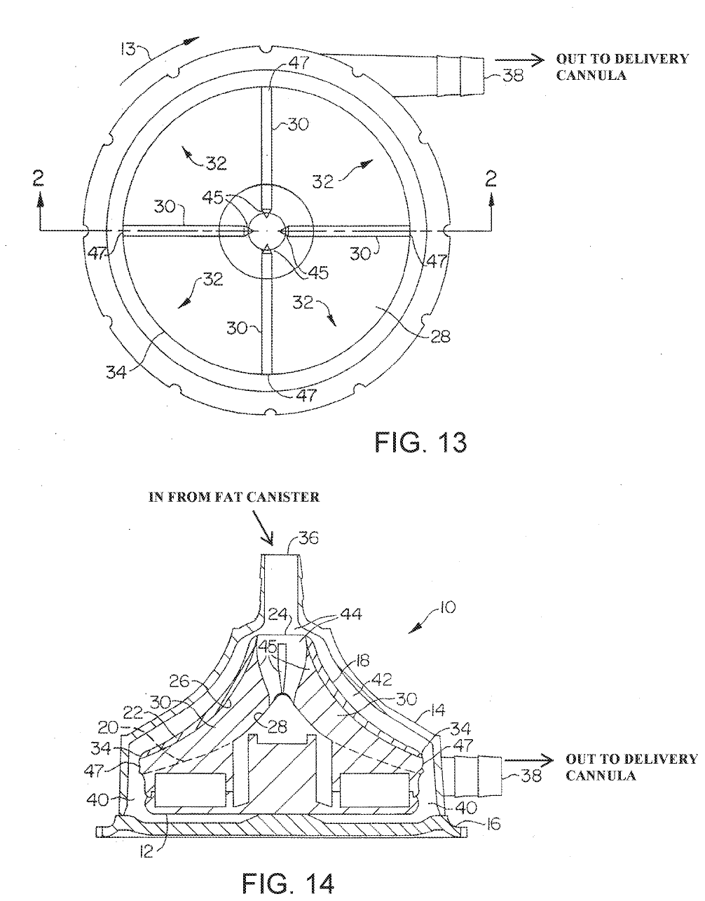

FIG. 1 is a perspective view of a high volume fat transfer system having a centrifugal pump. In this embodiment, a centrifugal pump is coupled to the fat canister and the delivery cannula via suitable tubing. In some configurations, the centrifugal pump is similar to pumps used to pump blood in configurations such as for left ventricular assist pumps (LVAD), or extra corporal blood pumps, or pediatric blood pumps. Various aspects of the design and operation of such pumps are provided in, for example, U.S. Pat. Nos. 6,264,601; 4,512,726 and 5,011,380, each of which is incorporated herein by reference in its entirety. In one aspect, the operating characteristics of the pump are adapted and configured using similar techniques as have been used with blood to optimize the pump to reduce, minimize or avoid damage to the fat delivered using the pump. In another aspect, the operating characteristics and designs are configured to enhance the mixing, distribution, infusion, smoothness or other characteristic of the fat to improve transfer, implantation and viability post-implantation. FIGS. 2 and 3 show two different representative centrifugal pump configurations for high transfer fat pumping operations as described herein. Centrifugal pumps may be single or multiple chamber in configuration. In some embodiments, pump inlets and outlets may be divided as in FIG. 2 or include cross over connections as in FIG. 3. Various other centrifugal pumps configurations are described below in FIGS. 13-39, 44 and 46.

In one aspect, the centrifugal pump is adapted and configured to provide controlled rate damage free or harmless pumping of harvested fat under conditions to overcome the line resistance in the fat pumping tubing and the fat delivery cannula for a desired fat delivery rate while also maintaining a desirous environment of fat transport. The desirous environment for damage free or harmless fat transport pump is one meeting one or more conditions that does not damage the fat, only induces minimal damage to fat, not clinically significant damage to fat or fat damage within a level that was produced using other transfer techniques or at a level where the result of healing process is sufficient to ameliorate incidental damage during pumping.

In still another aspect, the operating characteristics or design of the centrifugal fat transfer pump are adapted to impart no more damage or damage to fat within ranges similar to those of other fat handling, spinning, transport syringe or peristaltic based pumping systems. In still further configurations, while operating within a low fat damage range as described herein, the design or operating characteristics of the centrifugal fat pump may be further configured to provide desired levels of mixing or smoothing of fat prior to delivery to the patient. In still other configurations, the capabilities of centrifugal pumps to provide of oxygenation may be adapted for similar or modified infusion of gases or mixtures to aid in fat pumping, transport or viability.

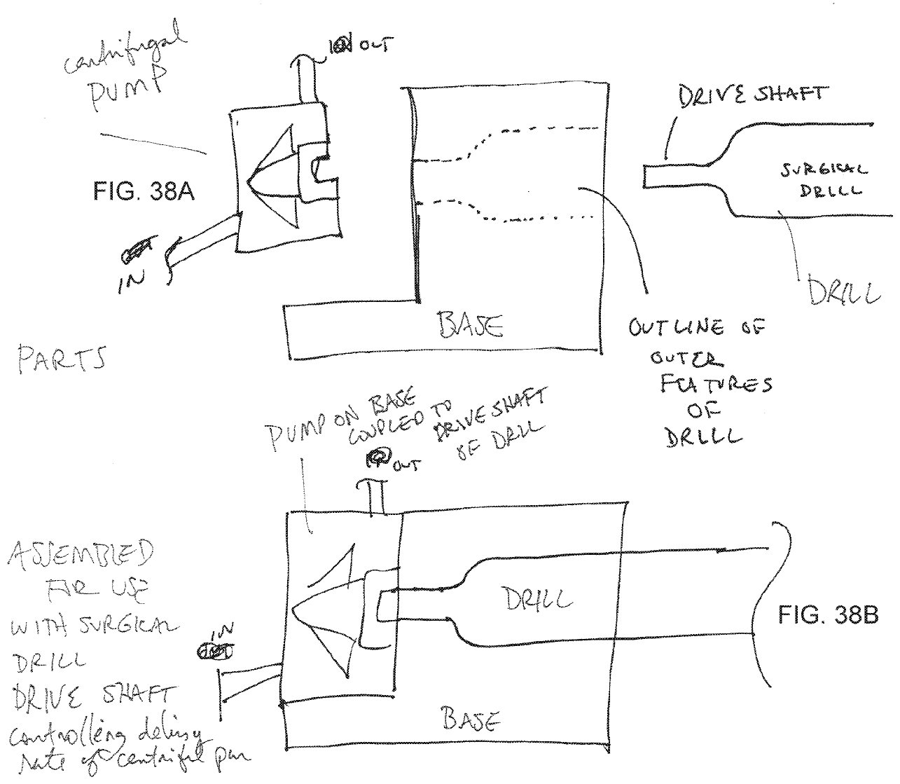

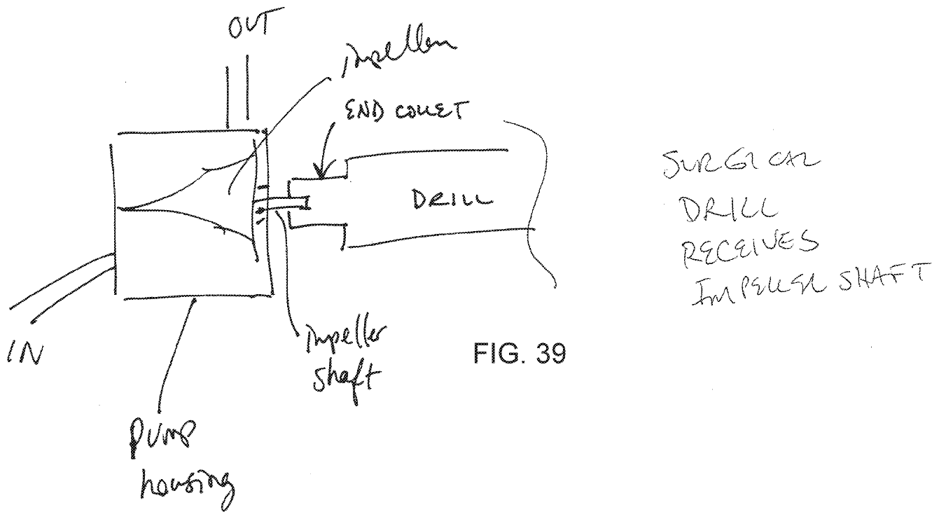

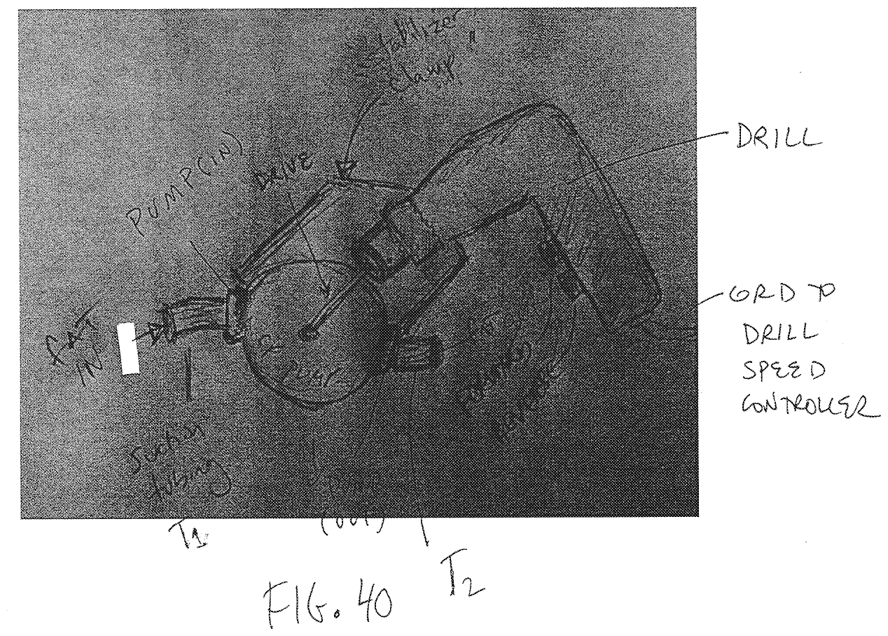

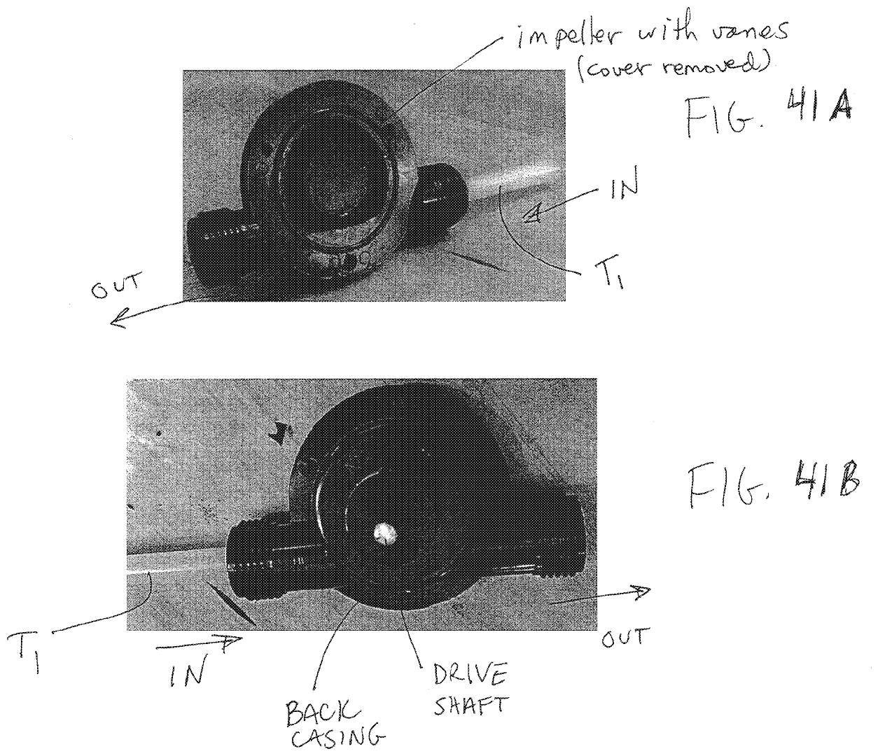

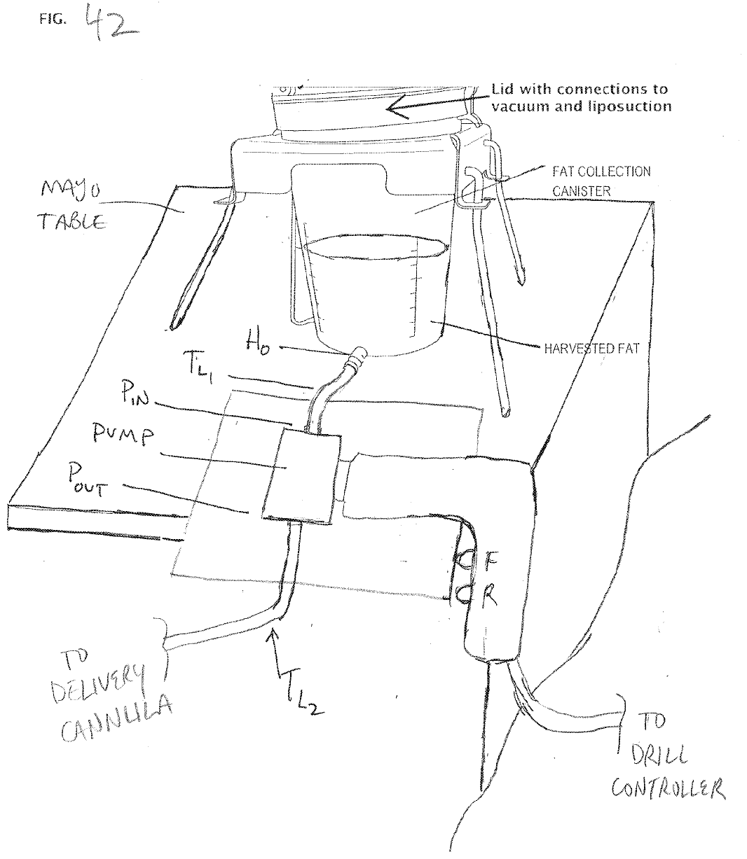

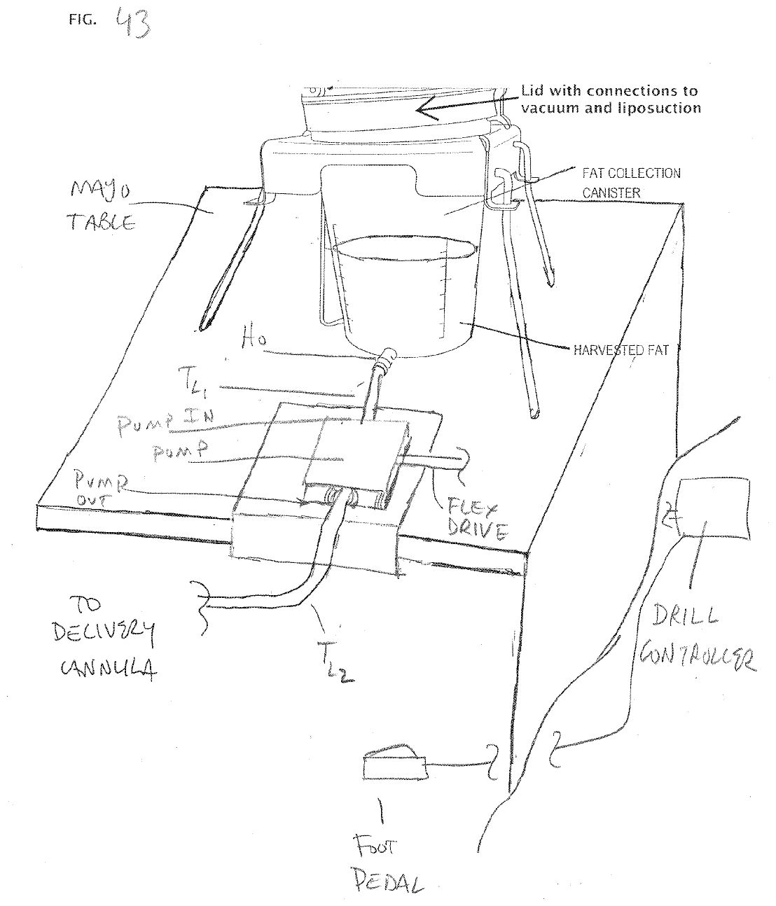

FIG. 4 illustrates a front view of a combined peristaltic infusion pump and centrifugal fat delivery pump. In use, the infusion side may be operated, controlled and monitored during preparation for harvesting and the centrifugal pump operated for fat delivery. One advantage of this configuration is that a single pumping unit may be needed in the operating room along with a fat harvesting canister to simplify preparations for high volume fat delivery while reducing the footprint and addition of equipment. The circuit illustrated in FIG. 1 may be modified to have this dual action pump unit in place of the centrifugal pump. Optionally, the end of the console may be adapted to receive and operate any of the centrifugal pump embodiments described herein, such as in FIGS. 38A, 38B and 39 or by using a magnetic drive, regular electrical connections, or a flex drive system. Additionally, an indication of pump output may be provided using a flow meter positioned at the pump outlet or along the outlet tubing or pump rotation speed and timing may be calibrated so that time of pump rotation provides an output indication to a user. In some embodiments, pump characterization tables and duration of pump operation may be used to estimate pumped fat volumes (see FIG. 45).

FIG. 5 is a flow chart of an exemplary closed circuit fat harvesting and transfer method 500. First, at step 505, there is a step of infusing a fat harvesting site of a patient with a tumescence fluid using a peristaltic pump. Next, there is a step of harvesting an amount of fat from the fat harvesting site into a fat collection canister. Next, at step 510 there is a step of connecting a first end of a first tube to an outlet of the fat collection canister and a second end of the first tube to an inlet of a centrifugal pump. Next, at step 515, there is connecting a first end of a second line to an outlet of the centrifugal pump and a second end of the second line to a fat delivery cannula. Next, at step 520, there is a step of operating the centrifugal pump to draw fat from the collection canister through the pump and into the fat delivery tubing to the fat delivery cannula into a fat delivery site on the patient. In one alternative embodiment, harvesting step 505 is performed using a cannula coupled to a handle that induces movement such as variable and controllable vibration or oscillation in the cannula. In another or the same embodiment, such handle may be used with the cannula used for fat delivery as in step 520.

Turning to FIG. 1 and step 505, there is a step of Fat Harvesting: This may be done through a variety of techniques but basically any standard or preferred liposuction cannulas, connecting the liposuction tubing to a canister reservoir with an outflow luer lock, such as the JAC Cell®, Revolve® by PALS Microaire®, or other sterile collection system.

In one specific embodiment, a method of harvesting and grafting fat included a super wet tumescence step whereby 2-3 cc of tumescence fluid was injected into the patient harvesting site for every planned 1 cc harvested fat. This embodiment is a further specific example of the performance of step 505 of the exemplary closed circuit fat transfer method 500.

The next step is processing.

This step may be done through the surgeons preferred technique, depending on the canister system used. If the JAC cell is used then the aqueous layer is allowed to decant and is first syphoned off through the luer lock connection. A warmer or heating element may be provided to maintain the harvested, separated fat at or near body temperature or to overcome cooling because of ambient conditions in the operating room. These and other variations are performed as part of harvesting step 505 which, like other steps of the exemplary method 500, are modified according to a number of practical factors, such as for example, the surgical skill and preferences of an individual surgeon performing the method as well as the clinical needs and circumstances of an individual patient receiving the benefits of the inventive method. These and other similar steps such as fluid injection and site preparation may be used to standardize or systematize fat fluid characterization as part of an overall pump characterization process is described in FIG. 45

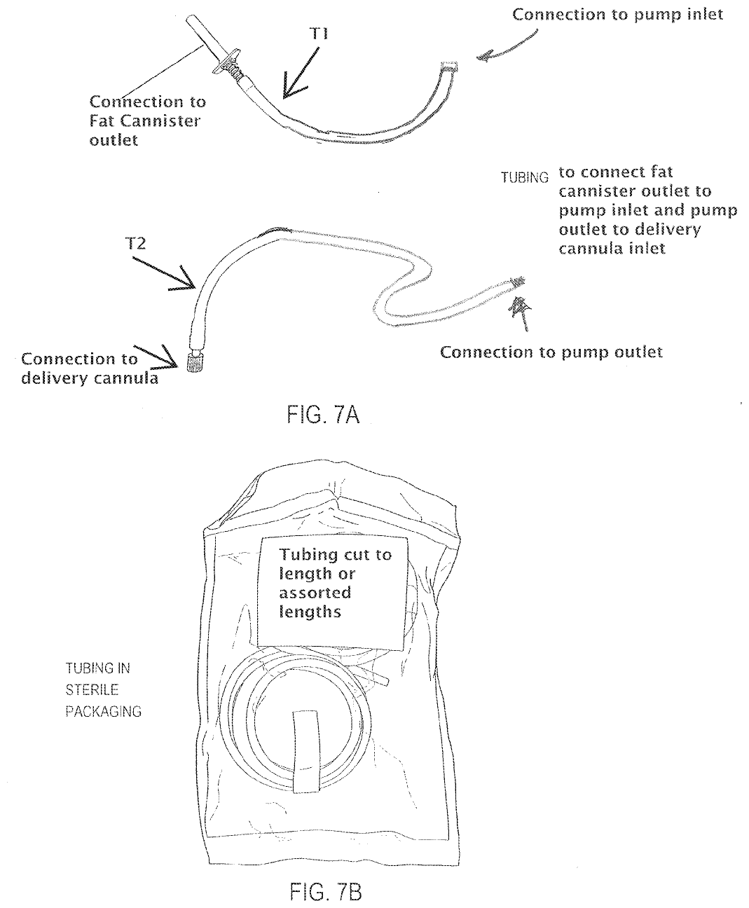

Next, a length of fat transfer tubing connects a first end of the tubing to the outflow valve of the fat collection canister. (see step 510) The second end of the tubing is connected to the pump inlet. Exemplary tubing is shown in FIGS. 7A and 7B, fittings may be provided based on the specific pump configuration used. Tubing with a slightly wider bore tubing works well without clogging, but other tubing as well can be adapted for connection to the pump. The two ends of the fat delivery tubing between the harvesting tank and the pump inlet remain on the operative field with sterility preserved. The tubing can be selected so as to be directly connected to the harvesting canister and to the pump inlet.

Next, at step 515, a length of the 2^{nd} tube is connected to the pump outlet and to the delivery cannula. as with the 1^{st} tube, both ends and the entire length of the 2^{nd} tube remain within the operative field with sterility preserved. The lengths of the 1^{st} and 2^{nd} tubes may be provided in precut lengths (FIGS. 7A and 7B). Optionally, tubing may be provided and cut to length based on user preference.

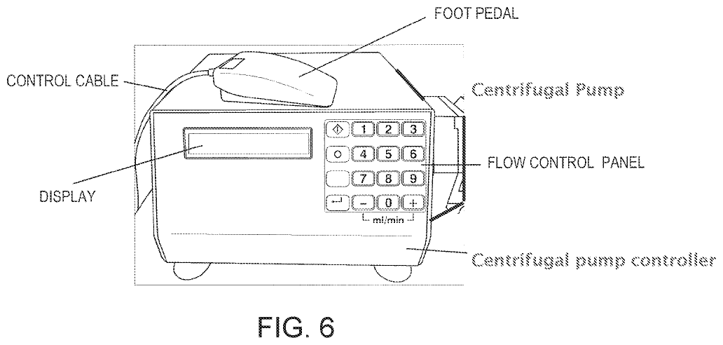

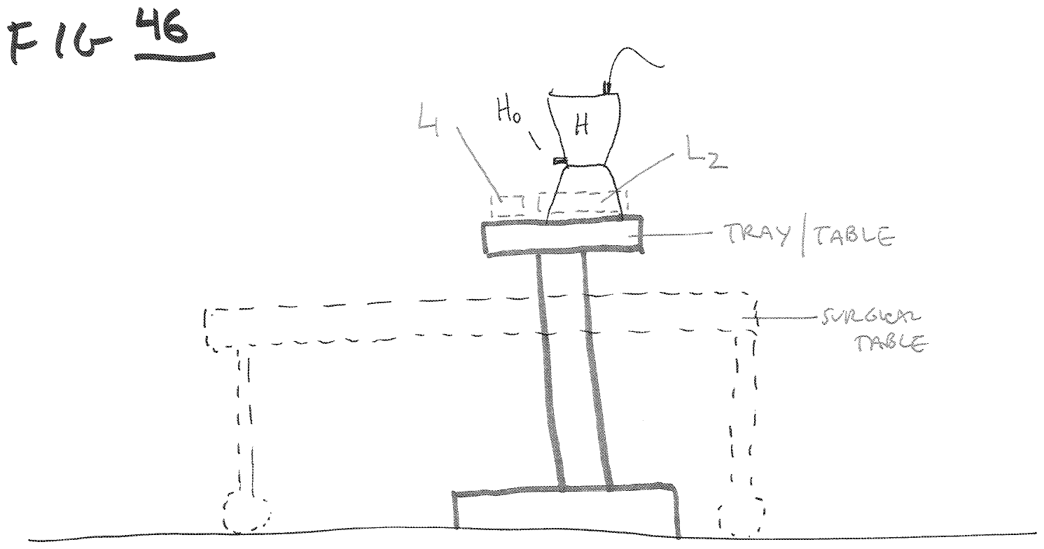

In another embodiment, the foot pedal of the centrifugal pump is used to provide flow control of the fat thereby leaving both hands free to address fat introduction pattern, palpate tissue turgor, contour and position of the recipient bed or breast. (see FIGS. 43 and 44). FIG. 6 is a perspective view of an exemplary centrifugal pump controller, foot pedal and control cable.

Once the delivery tubing is loaded, the pump and tubing appear as shown in the exemplary set ups of FIG. 1, 42, 43, 44, or 46.

Exemplary cases, indications and techniques for fat grafting using embodiments of the closed loop pump system are provided below.

1) Reconstructive Breast:

a) Partial defects

b) Radiated tissue

c) Implant Volume Inadequacy in higher BMI patients

2) Cosmetic:

a) Large volume—eg. Buttock augmentation

b) Small volume—Facial Rejuvenation

Exemplary Technique:

Harvest:

Tumescent Fluid (ringers lactate with 1 amp bicarb and one 30 ml bottle on lidocaine with epinephrine 1:100,000 dilution) is infused with Byron EZ Pump® with expected ratio to harvested fat of 2-3:1. PALS Microaire® hand piece is used for assisted liposuction harvesting. Harvest cannulas of 3, 4 or 5 mm are used to harvest the fat. Suction is provided using a Neptune® system with pressures kept below 300 mmHg.

Collection:

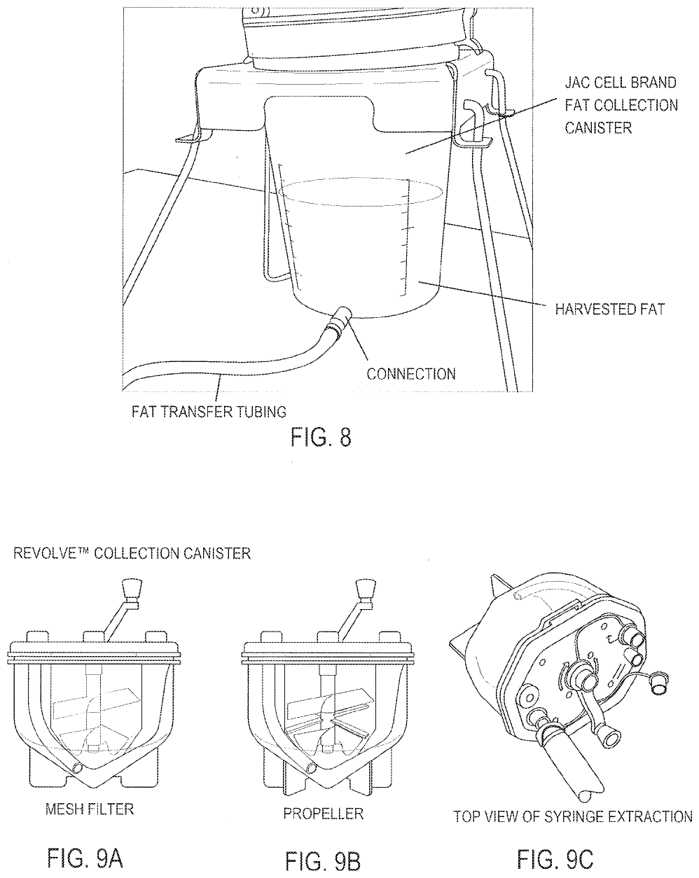

Fat is collected in either JAC Cell® or Revolve® collection systems. A JAC Cell® system is shown connected to an end of fat delivery tubing in FIG. 8. With the former gravitation separates fat from supernatant, with some supernatant left in canister (5-10%) to ease fluidity of transfer. With the Revolve the fat may be washed with ringer's lactate solution in the standard fashion.

Grafting:

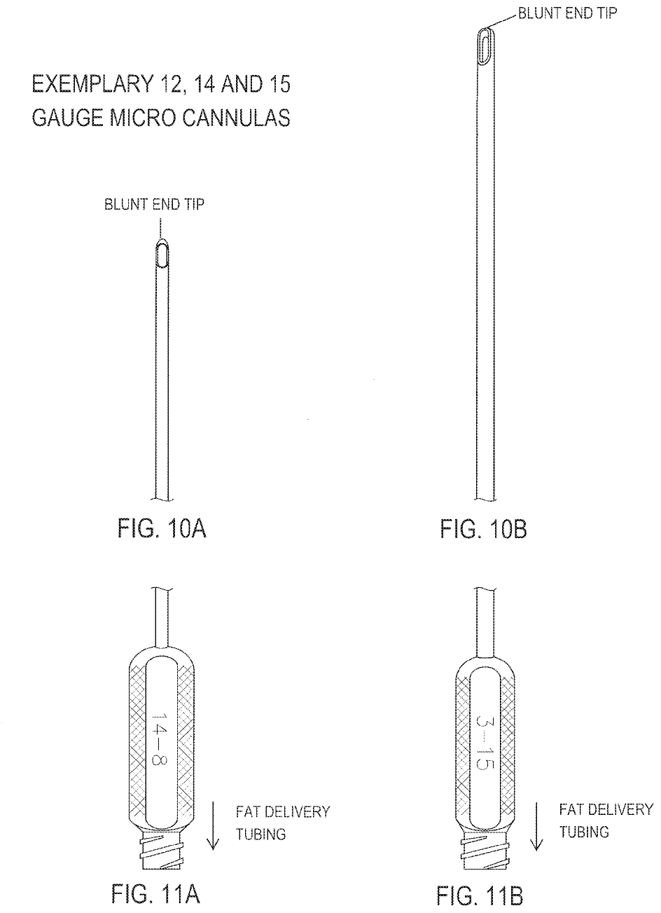

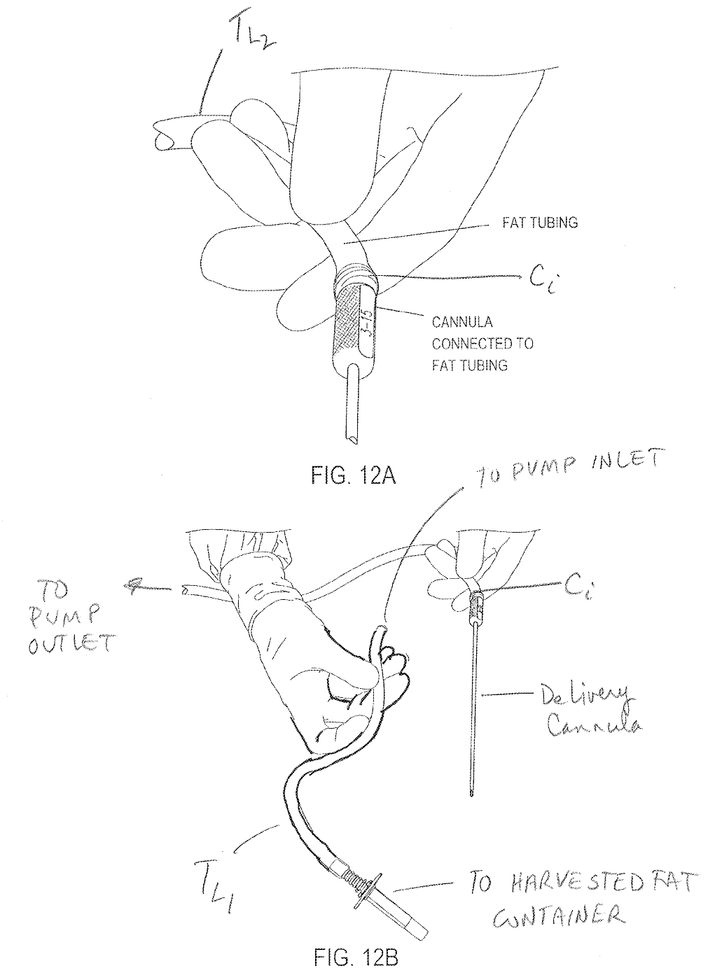

Tubing (TL1 and TL2) for the connections described above may be provided in preset lengths with fitting (if needed) as shown in FIGS. 7A and 7B. The length of the 1^{st} and 2^{nd} tubes is optimized to minimize pumping length (i.e., distance fat must travel within tubing). The position of the pump inlet relative to the harvest canister outlet will determine the appropriate length of the first tubing. The second tubing length is selected to provide enough maneuvering room to reach the patient, which may vary with operating room set up dimensions, and user preferences. The other tubing end is then fit onto the preferred injection cannula. Exemplary injection cannulas are 12, 14 or 16 gauge microcannulas. Various views of exemplary cannulas are provided in FIGS. 10A, 10B, 11A, 11B, 12A and 12B. In some instances, a clamp may be attached over the tubing end to microcannula joint to prevent accidental blow off of the tubing from the cannula and possible loss of fat. (See FIG. 12A) In some pump embodiments, a foot pedal is provided to control flow. Flow rates may be adjusted using pump controls or via foot pedal. In one aspect, fat delivery is initiated at 100 ml/min. The pumping rate may be adjusted depending on fluidity of fat which may change according to the bore of the harvest cannula, the amount of supernatant left with the fat, room temperature or other factors. (See FIG. 45) In some aspects, one or more additive components may be incorporated into the harvest site or harvested fat in order to improve flow characteristics, graft viability, or other factors to improve closed loop fat transfer.

Sitting the patient upright, with all standard and proper protections is critical to assess contour changes prior to and during fat transfer. In these exemplary cases patients have both arms completely wrapped in foam and arm boards held out at 40 degree angles to the bed, and are also secured with a standard belt to the bed.

### Alternative Centrifugal Pump Configurations

One embodiment of a centrifugal pump of the present invention is illustrated in FIGS. 13 and 14. FIG. 13 is a top view of a pump 10. Pump 10 has been described previously with respect to the pumping of blood it is believed that modifications to designs and operation such as lower rpm for flow rates of the high volume transfer of harvested fat will make the pump embodiments equally effective for pumping fat in the procedures described herein.

FIG. 14 is a cross-sectional view of pump 10 taken along line 2-2 of FIG. 13 through the axis of rotation of the pump rotor 12. Rotor 12 rotates in the direction of arrow 13. In addition to rotor 12, pump 10 includes pumping chamber 14 which may, as in this embodiment, be constructed of a transparent material, and base 16 which is essentially circular. Rotor 12 includes a shaft (not shown), and an impeller 18 having a lower section 20 and an upper section 22 which, in this embodiment, is made of a transparent material to allow the fat to be viewed as it passes through the pump. Upper section 22 is provided with a central opening 24. Upper section 22 and lower section 20 have oppositely disposed surfaces 26 and 28, respectively, which are separated by a plurality of separating surfaces 30. The dimensions of the sections and spacing of the sections may be adapted to the characteristics of the fat being handled by the pump.

In this embodiment, separating surfaces 30 are integrally formed as a portion of lower section 22 although it should be realized that they could be formed as separate pieces or as an integral portion of upper section 22. Alternatively, impeller 18 could be formed of integral construction in any conventional manner.

Separating surfaces 30 divide the space between oppositely disposed surfaces 26 and 28 a plurality of fixed chambers or flow passages 32 which extend radially from central opening 24 to the peripheral edge 34 of impeller 18. Harvested fat from the canister enters pump 10 through an axial inlet 36 and is discharged through a tangential outlet 38 into tubing and the delivery cannula. Inlet 36 and outlet 38 maybe located elsewhere in other embodiments. The locations shown in this embodiment are believed to have the potential for suitable pump efficiency and fat handling characteristics.

The pump rotor 12 may be driven in any conventional manner such as by the magnetic coupling of rotating drive magnets located in a controller console and driven magnets embedded in rotor 12. Such a drive system is well known and utilized commercially, for example in the BioMedicus, Inc., BP-80 blood pump with lower rpm operating speeds and lower volumes of fat pumped as set forth herein. Optionally, the rotor may be driven using a drill or other external drive shaft as detailed in FIGS. 38A, 38B and 39.

During pump operation, harvested fat enters the pump through inlet 36, Direct or indirect drive means coupled to rotor 12 causes impeller 18 to rotate. Fat enters central opening 24 and is caused to move downwardly and outwardly through flow passages 32 by the centrifugal action of the pump and the forces exerted upon the fat by oppositely disposed surfaces 26 and 28 and separating surfaces 30. The fat is collected in an unobstructed annular chamber 40 located between impeller 18 and housing 14. Some of the fat is then discharged through outlet 38.

A portion of the fat collected in annular chamber 40 is not discharged through outlet 38 but flows back towards inlet 36 in the space 42 between the upper section 22 of impeller 18 and housing 14. This backflow of fat is useful if air bubbles which have been inadvertently introduced into the fat during harvesting or handling or which have formed in the fat are carried along with the back flowing fat towards inlet 36. Even if air is not present, this back flow of fat may be beneficial to improving fat characteristics or to allow improved texture and mixing of fat for more even textures when implanted. If present, air bubbles collect in an air entrapment pocket 44 located near inlet 36. The precise location at which air is entrapped is dependant upon the position of the pump during use and upon the pressure differential between the inlet and outlet of the pump. Depending upon desired operating characteristics for fat pumping, the pressure differential may be large with the pressure at the outlet being greater than at the inlet. A large pressure differential enhances the air entrapment capabilities of the pump since any bubbles which are pumped with the fat through flow passages 32 are forced along with the back flowing fat in space 42 to the area of low pressure near the inlet. Flow passages 32 are constructed to maximize the inlet to outlet pressure differential and thus increase the air entrapment capabilities of the pump. In one aspect, the pump is modified by extending the leading edge 45 and trailing edge 47 of separating surfaces 30 generally the entire distance from central opening 24 to peripheral edge 34. This maximizes the length of the fixed chambers or flow passages 32. Since the pressure differential between inlet and outlet increases as the length of the flow passages 32 increases, this construction maximizes the pressure differential thus enhancing the air entrapment capabilities of the pump. By varying the length of the flow passages 32 the pressure differential can be changed in order to tune the pump to any desired harvested fat flow dynamics. In one aspect, this may be done by shortening the length of separating surfaces 30 such that trailing edge 47 is moved closer to the leading edge 45 and central opening 24. This tuning procedure may be used for all of the impeller variations shown herein and is not illustrated in the drawings. It is important to note that the pump should not be tuned by altering the position of leading edges 45. Leading edges 45 are positioned generally at central opening 24 in order to reduce the velocity of the edges with respect to the fat entering the pump from inlet 36. If separating surfaces 30 are shortened by moving leading edges 45 away from central opening 24 the velocity of the leading edges 45 with respect to the entering fat will be increased. This increased velocity may create turbulence within the harvested fat that may increase the risk of shear or other damage to the components of the harvested fat.

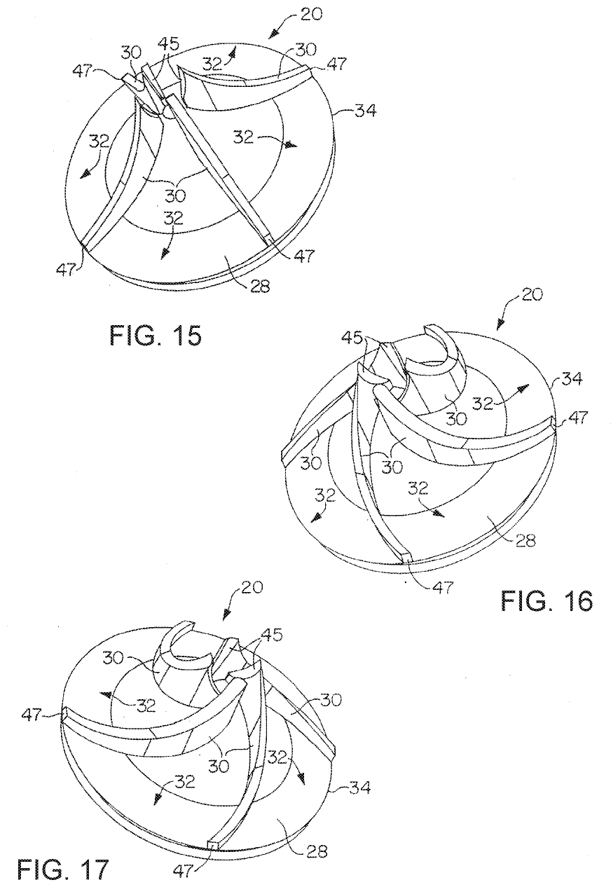

FIG. 15 is a perspective view of lower section 20 of impeller 18. Separating surfaces 30 radiate straight out in a linear fashion in a direction from the axis of rotation to peripheral edge 34 to form flow passages 32 between lower section 20 and upper section 22 (not shown in this figure).

FIGS. 16 and 17 show alternative designs for lower section 20 of impeller 18.

In FIG. 16 separating surfaces 30 radiate spirally in a direction opposite to that of the flow of fat through tangential outlet 38. In FIG. 17 separating surfaces 30 of lower section 20 radiate spirally in the direction of the flow of fat through tangential outlet 28. In this variation the fat flowing through flow passages 32 is urged by partitioning surfaces 30 to flow in the direction of the fat being discharged through tangential outlet 38 thus further improving pump efficiency. The pump of these various embodiments of the present invention is able to pump harvested fat in a gentle yet efficient manner. Separating surfaces 30. which extend generally from the central opening 24 of upper section 22 to peripheral edge 34 impart an impelling force on the fat with an efficiency exceeding conventional vaned pumps. This is done, however, with the added advantage of the elimination of the low pressure turbulence areas which existed in the area behind the vanes in conventional vaned pumps. These low pressure turbulence areas are eliminated by enclosing the flow paths between the upper section 22 and lower section 20 forming flow paths 32. The dimensions of the flow paths may be adjusted to correspond to harvested fat characteristics and other factors related to handling fat prior to implantation back into the body. Since this design does not allow flow over the top of separating surface 30, no low pressure turbulence region is created. Thus, it is believed that the fat is handled with less damage or lower likelihood of damage which is balanced by the advantages to a shorter surgical time detailed herein. Additionally, by extending the leading edge 45 of separating surfaces 30 substantially all the way to central opening 24, the velocity of leading edges 45 with respect to the entering blood is minimized thus further enhancing the gentle blood handling characteristics of the pump. In some embodiments, this construction also increases the ability of the pump to trap air since the pressure differential from the inlet to the outlet is maximized.

Although not shown in the figures, the shape, number and arrangement of separating surfaces 30 may be varied. Thus, the cross-sectional configuration of flow passages 32 could be formed in a variety of shapes including rectangular, circular or oval.

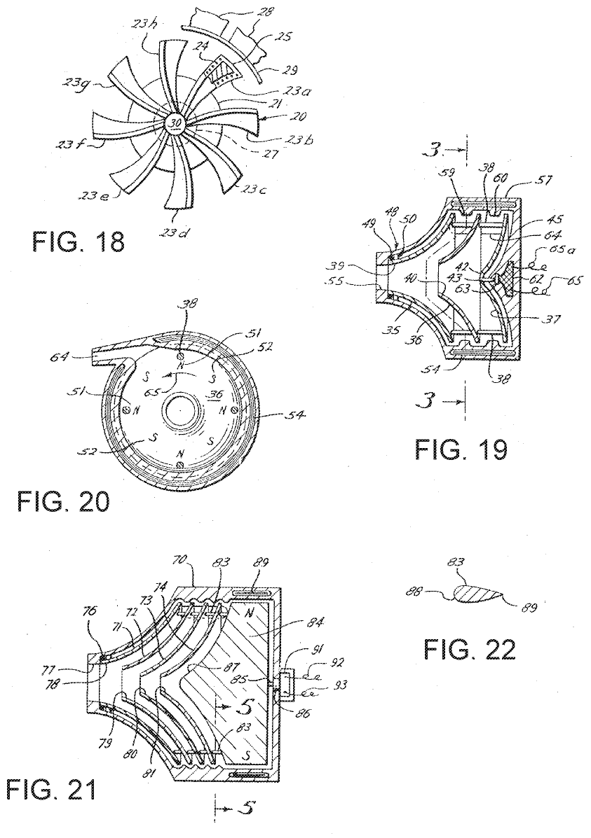

The pumping action obtained by the embodiments of FIGS. 13-17 may be described as radially increasing pressure gradient pumping, or in some cases more specifically as forced vortex radially increasing pressure gradient pumping. In centrifugal pump designs of this type, the fluid acted on by the vanes of the impeller is positively driven or thrown outwardly (radially) by the vane rotation. The fluid as it moves from the vanes to the ring-shaped volute space beyond the tips of the vanes is reduced in velocity, and as the velocity decreases the pressure increases according to Bernoulli's theorum. On the other hand, in the pump configurations provided according to FIGS. 18-32, the pumped fluid is not driven or thrust outwardly but instead is accelerated to circulate in the pumping chamber at increasing speeds as it moves farther and farther from the center. At the outer periphery of the accelerator or rotator, the speed of the fluid is maximum.

The action of the fluid in the pumps of FIGS. 18-32 may be clarified by analogy to a glass of water turning about its vertical axis without sideways motion or wobble. Because of its contact with the sides and the inherent potential shear force of the water in the glass, the water will rotate, in the form of a forced vortex, without much slip or shear between radially adjacent particles of water, and the water radially away from the center of rotation will be moving faster than water nearer the center. If water is introduced through a tube at the axis of the glass and water is removed through one or more holes through the side of the glass, water will be pumped by the rotation of the glass. As a result, in the pump rotators provided in FIGS. 18-32 as well as in a number of different forms are designed such that they act to increase the swirling speed of the liquid passing through the pump, but do not act to drive or throw the liquid toward the periphery or volute of the pump chamber, but instead only increase the rotational speed of the liquid. As the rotative speed of the liquid is increased, it achieves a higher “orbit” about the center of the accelerator and moves toward the periphery of the chamber. It is believed that characteristics of harvested fat such as density and viscosity may enhance the benefit of these types of rotors.

In each case, the pumps of FIGS. 18-32 are provided with internal driving means in the form of an electric motor drive of some form. The electrical parts of the electrical motor drives are presented in several different forms as will be later further described. Alternatively, the pumps of FIGS. 18-32 may be driven by an external magnetic drive as described herein or using a drill or drive shaft (see FIGS. 38A, 38B and 39).

Referring first to FIG. 18 of the drawings, a pump rotator 20 is shown which has a plate or disc 21 at each of its sides, which may be identical or of different forms or sizes, only one being shown in the drawing, and between which there are provided the equally circularly spaced curved blades 23a-23h, each curved from its inner end to its outer end as shown and each having a twist throughout its length similar to the twist of the propeller. The blades or vanes may extend beyond the outer edges of the disc 21. Each blade 23a-23h has therearound a winding 24, which is covered by an impervious layer or membrane 25. The blade windings are connected to contact elements of a commutator 27. The surrounding pump housing is provided with the circularly spaced magnets or coils 28, which are separated from the pumping chamber by an impervious layer or membrane 29. The commutator rotates with the rotator in the usual manner of an electric motor. The rotator windings and housing magnets or coils constitute an internal electric motor for driving the rotator to pump fluid. The electric motor thus provided may be of any of the known types, AC or DC, with or without commutation, powered by electrical conductors leading thereto from any suitable AC power source or from a battery or from an external magnetic drive.

While the self-contained drive motor is herein shown and described in connection with the form of rotator shown in FIG. 18, it will be understood that it may be provided in conjunction with other forms of rotators disclosed herein or in said U.S. Pat. No. 3,487,784. Teflon may be used for the membranes 25 and 29 covering the windings of the electric motor structures or as an outer layer for any fat contacting surface.

Accelerator or rotator 20 may be mounted for rotation within any suitable pumping chamber or housing. The chamber or housing is not shown in FIG. 18 of the drawings. Harvested fat entering the pumping chamber adjacent the shaft 30 on which rotator 20 is mounted is moved circularly by the vanes of the rotator to move progressively toward the outer peripheral portion of the pumping chamber. An outlet of any suitable form is provided for exiting of the fluid from the periphery of the pumping chamber. The fluid inlets and outlets may be as described in said U.S. Pat. No. 3,487,784 and adapted for connection to surgical tubing or connection to other components as described herein.

Referring now to FIGS. 19 and 20 of the drawings, a plurality of flared rotator vanes 35, 36, 37 are connected together with plural rods 38 spaced about the vanes. Vanes 35 and 36 have circular openings at their smaller ends designated respectively by reference numerals 39, 40. Vane 37 has a convex rounded center 42 which is connected to a shaft or rod 43 which is rotatively disposed through an opening of housing end portion 45. Housing portion 45 is inwardly curved corresponding to the curvature of vane 37 and the vane is spaced therefrom as shown. The smaller end of vane 35 outwardly of opening 39 thereof is rotatively sealed within the housing 48 by a circular seal 49, for example, an O-ring, and a bearing 50 is provided adjacent the seal. Housing 48 is inwardly curved corresponding to the curvature of the vane 35.

The three vanes rotate together when driven in rotation as will be described. The vanes 35-37 are each permanently magnetized to have alternate magnetic north and south poles “N” and “S” spaced therearound as indicated in FIG. 20 by reference numerals 51, 52, respectively, of vane 36. The other two vanes have magnetic poles therearound in positions corresponding to the pole positions shown for vane 36. The windings of the electrical motor assembly are indicated by reference numeral 54, and are disposed within housing 48 around the outside of the outer edges of vanes 35-37. Separate magnets may be connected to the vanes, instead of the vanes being magnetized, if desired.

Housing 48, at its left-hand end as shown in FIG. 19, is inwardly and outwardly curved corresponding to the curvature of vane 35. An inwardly projecting thickened wall portion 55 surrounds the fluid inlet to the pump. Portion 45 of the housing is, as has been described, inwardly curved corresponding to the curvature of vane 37, and is flat at its outer side. The surrounding portion 57 of the housing wall has winding 54 disposed therein and extends around the outer edges of the vanes, and is spaced outwardly uniformly therefrom. The inner surface of wall 57 has a pair of circular bevelled-sided annular projections 59, 60 which are centered between the vanes 35, 36 and 36, 37 respectively.

The electrical switching elements for the windings 54 are disposed inside of housing portion 45 at 62. The rod 43 is journaled through bearing 63 and serves to rotate the necessary switching elements at 62. The switching elements 62 are connected to the windings in customary fashion, these connections not being shown in the drawings because they are of standard conventional form in order that the winding currents may be alternated as required for the apparatus to perform its rotative motive function. An electric power source for the drive motor is provided and is connected to leads 64, 65, the power source being of any convenient nature. When electrical current is supplied to the windings through switching elements 62, the magnetic rotators rotate. Fluid enters the pump through the opening within thickened wall portion 55 and flows between vanes 35 and 36 and between vanes 36 and 37, reaching these areas through vane openings 39 and 40. Since there are no rotator surfaces to directly force the fluid radially outwardly, as in a conventional centrifugal pump, friction between the vanes and the fluid causes the fluid to commence circulating round and round in circular fashion, and gradually moving outwardly toward the pumping chamber periphery. The housing has an outlet 64 shown to be more or less tangential of the chamber periphery, but which may be directed in any flow direction from the chamber. Fluid the velocity of which has been increased by rotations of the vanes is caused to move under pressure out of outlet 64 to accomplish the pumping function of the pump. Arrow 65 indicates the direction of rotator rotations.

Referring now to FIGS. 21 and 22 of the drawings, the housing 70 is of the same general form as housing 48 of FIGS. 19 and 20. A plurality of vanes 71-74 are disposed within the pumping chamber, the outermost vane 71 being rotatively sealed by a circular seal 76, for example, an O-ring, and journaled in a suitable ring bearing. The housing has a thickened wall portion 77 around the fluid inlet to the pump and the successive vanes 71-74 have progressively smaller circular flow passages 78-81 affording fluid flow to between the spaced apart vanes. The vanes are connected by circularly spaced elements 83 to a rotating magnet body 84. Body 84 carries a concentric shaft 85 which is journaled in bearing 86. The end 87 of magnet body 84 is flaringly curved corresponding to the curvature of vane 74 and is spaced therefrom by a distance about equal to the vane spacings. Magnet body 84 has therearound alternating north and south poles permanently magnetized therein, similarly to the magnetic poles of the vanes 35-37 of FIGS. 18 and 19. The connection elements 83 are of streamlined cross section as is best shown in FIG. 22. The vanes are larger and rounded at one edge and tapered to a thin edge 89 at their opposite sides. The rounded edges 88 of the elements 83 are the leading edges which are impelled through the fluid being pumped and the shapes of the elements provide streamline flow therearound whereby turbulence is not caused by the elements 83. The pump of FIGS. 21-22 has imbedded in housing 70 the windings 89 similarly as the windings 54 are provided in FIGS. 18-19. The electrical switching elements for the windings are disposed in a chamber 91 which is provided at the end of rod or shaft 85. The electrical connections between the switchings elements and the windings are not shown as they are conventional in nature. Electrical power is supplied through electrical leads 92, 93, from a suitable power supply, not shown.

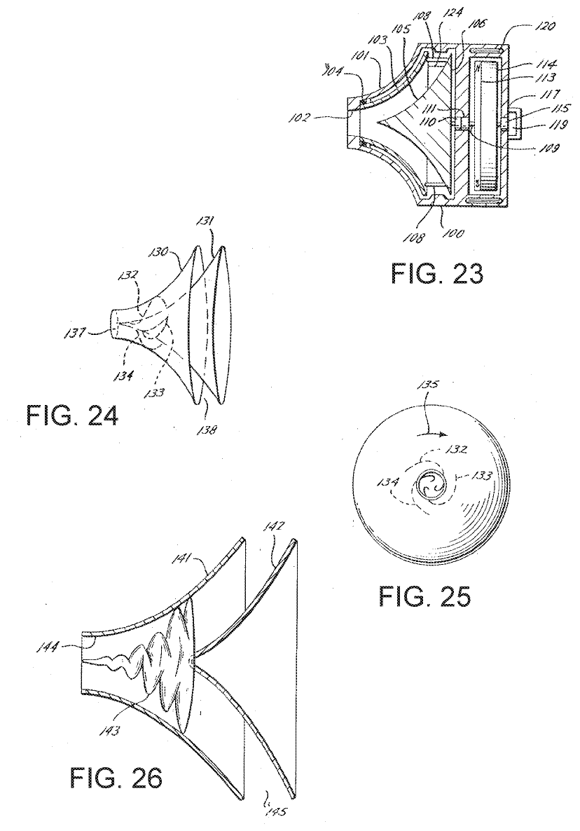

Referring now to FIG. 23 of the drawings there is shown a motor-pump structure having a partitioned housing, the rotator elements being disposed within one chamber of the housing and the magnetic armature element being disposed in a separate chamber. The housing 100 of the pump shown in FIG. 23 is circularly and curvingly flared at end 101, inwardly and outwardly. A thickened wall portion 102 surrounds the circular fluid inlet to the pump. A circular flared vane 103 is sealed and journaled to the pump housing at 104. A rotative vane member 105 is circularly and flaringly formed and spaced from vane 103. The opposite side 106 of vane 105 is circular and flat as shown. The vanes are connected together by a plurality of circularly spaced rods 108. A rod or shaft 109 is concentrically connected to vane member 105 and is journaled through bearings 110, 111. At its other end, rod or shaft 109 carries a circular disc magnet 113 having spaced therearound alternating north and south poles of permanent magnetism as previously described for the other embodiments. The magnetic disc 113 is affixed flatly against a thicker disc 114 which rotates therewith. Rod 109 extends into a terminal bearing 115. Housing 100 is flat at its end 117. A chamber 119 formed on the end 117 of the housing contains the electrical switch elements for the motor winding 120 which is disposed as before within a circular annular chamber of the housing, outwardly surrounding the magnetic disc 113. Upon supply of electrical power to the motor windings 120 through the switching elements 119, the magnetic disc 113, disc 114 connected therewith, and the rotator elements 103, 105 rotate to pump fluid entering through the passage within thickened wall portion 102 to flow within vane 103 and outside of vane 105. The pump, again, has no impeller surfaces causing fluid thrust radially outwardly within the pumping chamber so that the fluid is caused to rotate circularly with constantly increasing radius, to be expelled through outlet 124 from the housing. The outlet 124 may be designed similar to outlet 64 of the FIGS. 19-20 embodiments.

In FIGS. 24-25, and FIGS. 26-32, there are shown various forms which the rotators or accelerators for pumping of the fluid may take. The rotators are shown more or less schematically in the drawings. In FIG. 24, two circularly flared pumping rotators 130, 131 are connected by spiral shaped connectors 132, 133 and 134, with vane or rotator rotation in the direction indicated by arrow 135. The spiral connectors 132-134 serve to institute circular flow of the fluid passing between the vanes as the fluid enters through opening 137. The vane rotation causes the fluid to flow in increasing radius circles to exit at 138, the circular space between the outer vane edges.

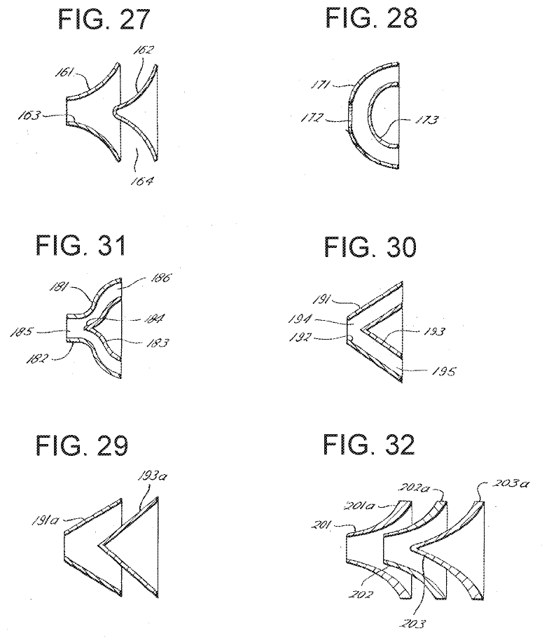

In FIG. 26, there is shown a rotator assembly including trumpet shaped circular flared rotators 141, 142 which are connected by a connector 143 of the form of an increasing amplitudes screw formation. As fluid entering between the rotators at opening 144 of vane 141 is brought into contact with connector element 143, the screw shape of the connector impels the fluid to circular flow, the flow continuing outwardly between vanes 141, 142 to exit from therebetween at 145.

The vanes in FIGS. 27-32 are of various shapes to indicate rotator surfaces which are useful in circularly impelling the fluid for pumping within a pump of the type herein described, the respective pumping chambers (not shown) being of corresponding interior shape. In FIG. 27 the vanes 161, 162 are respectively of trumpet shape, circularly flared, vane 161 having an inlet opening 163, and fluid is caused by friction with the vanes to move in circles of increasing radius to exit finally between the outer vane edges at 164. In FIG. 28 the vanes are of hemispherical hollow form, the outer vane 171 having an inlet opening 172, and the vane 173 being of continuous spherical shape. In each case, the vanes are connected together by appropriate rods or other connector elements such as have been described. The vanes are mounted within a pumping chamber with appropriate seals and bearing surfaces around the flow inlet and with appropriate supports for suitable rotations of the vanes.

Referring now to FIG. 29, the vanes shown are of inconstant curvature, the vane 181 having a cylindrical tubular inlet 182 and the vane 183 having a more or less pointed formation 84 to direct the circularly moving fluid to between the vanes without friction, the fluid entering at 185 and exiting at 186. In FIG. 30 vane 191 is of truncated conical form, having an inlet at its apex 192, and the vane 193 being of conical form. Fluid enters at 194 and is expelled at 195.

The vanes 191a and 193a are similar to those shown in FIG. 30, except that the conical angle of vane 193a is flatter than the conical angle of vane 191a, and the vanes are of the same outer diameter, as shown. In FIG. 32, there are shown three vanes which are of the circular flared form heretofore encountered, each vane being thin walled at its inner portion 201, 202 and 203, respectively, and relatively thicker walled at its outer radial portion 201a, 202a, and 203a, respectively.

It will be noted that in all of the embodiments shown and described, the fluid inlet through the housing wall and the initial vane into the interior of which the fluid passes, are smoothly merged so that there are not abrupt changes of fluid flow therepast. In each case, the fluid to be pumped flows inwardly between rotating accelerator vanes or rotators, to be driven by friction with the rotators in a circular flow direction. The pumps operate on a forced vortex principal, there being no impeller surfaces in the pumps for impelling blood or other fluid material being pumped radially outwardly toward the periphery of the pump chamber. A forced vortex pump operates on the principal that a rotating chamber causes rotation of its contents, with creation of a vortex, so that a body of circulating fluid is maintained within the pump chamber by rotation of the rotator vanes at opposite sides of the chamber. The rotational speed of liquid in the pump is increased from the center to the periphery of the pumping chamber. The liquid is withdrawn at the peripheries of the vanes.

It is believed that fat pumped utilizing the pumps of FIGS. 18-32 is not submitted to any substantial agitation by the rotation of the vanes, or by any other portion of the pump apparatus. There are no sudden changes in direction of flow through the pumps, all joints between surfaces being smooth and all surfaces over which the fluid flows being smooth. Where there are more than two vanes, there are more than two spaces in which the fluid is rotated and pumped. The embodiments of FIGS. 18-32 provide gentle, nonturbulent handling of the pumped fluid, as is illustrated by the aforementioned rotating glass of water with nothing to rotationally accelerate the water but the smooth side of the glass. Yet, the water after a time rotates with the glass and continues the rotation as long as the glass continues to rotate.

It will be realized that pumps may be supplied according to the invention with any number of pumping stages, and may include individual pumping stages of any of the types mentioned herein, and in any combination.

In the case of each of the pumps and rotators shown in the drawings, it will be noted that the rotators are designed to avoid turbulence and to avoid rapid pressuring and depressuring of the harvested fat being pumped and also to avoid any physical grinding or abrasive action upon the fat. As has been made clear, these rotator designs are made in this manner in order that harvested fat being pumped, some containing solids in suspension, will not suffer detriment and will not be destroyed by the pumping operation.

It is believed that the revolution speeds permitted of the rotators employed with the pumps of FIGS. 18-32 are kept minimal. The several rotator designs presented are each of a form adapted to progressively increase the circular fluid velocities as the rotator turns and as the fluid advances toward the periphery of the rotator. In each pump presented, an annular fluid circulation space is provided which is entirely unobstructed and regular so that fluid can circulate therein without turbulence or baffle effects.

Referring to FIGS. 19 and 20 of the drawings, the plural rods 38 spaced about the vanes can be eliminated and the rotators 35 and 36 levitated within the electromagnetic field generated by the windings of the electrical motor assembly indicated by reference numeral 54. The rod 43 can also be eliminated and all rotators 35, 36, and 37 would then be levitated in the electromagnetic field generated by the windings of the electrical motor assembly 54. The levitated rotators 35, 36, and 37 can all revolve at the same speed of rotation or each can revolve at a rotational speed independent of the others. Plural rods 38 or connecting elements 83 (this latter illustrated in FIG. 22) may be used to join two or more rotators while the rotators are levitated in the electromagnetic field generated by the electrical motor assembly 54. In instances in which multiple rotators are levitated in an electromagnetic field, two or more rotators may be connected by plural rods 38 (illustrated in FIG. 20) or connection elements 83 (illustrated in FIG. 22) while one or more additional rotators are levitated and revolve without being connected to a neighboring rotator. It is understood that operation and levitation of the rotators within an electromagnetic field as described can be applied to the various forms of rotators disclosed herein or in said U.S. Pat. No. 3,487,784.

The pumps and their parts may be constructed of any materials compatible with their intended use, including metals, mineral materials, plastics, rubbers, or other suitable biocompatible materials. A consideration must be given to biological compatibility so that trauma to the pumped, harvested fat will not result. In one aspect, Teflon, PTFE or FEP may be used at a coating on all fat contacting surfaces. Still further, noncorrosive metals and alloys that are biocompatible with handling fat may be used in the pumps where required. Additionally, the pump housings and rotators or impellors may be constructed of suitable material so that the housing may be rigid, semirigid, or elastic in whole or in part. In some aspects, nonrigid components or constructions can be used for imparting pulse configurations to fat in the pump to provide a way to pause fat flow during delivery or modify or further control fat flow rates during delivery depending upon the needs of an individual patient or the clinical need.

While the rotators shown herein may in some cases perform better when rotated in one direction, it should be understood that they may be rotated in either direction i.e., reversed, without other modification of the pumps. Each of the rotators presents surfaces to the fluid being pumped, to cause accelerating circular fluid motion in the pumping chamber. In some cases, the surfaces are parallel to the fluid flow; in other cases parallel and nonparallel surfaces are provided. Each of these surfaces, of whatever form, will accelerate the fluid regardless of the direction of rotation of the rotator. Each rotator should be rotated at a speed such that essentially no fluid turbulence occurs, and differences in the rotator designs affects the maximum speed at which a particular rotator may be rotated. The physical and flow properties of the fluid pumped will, of course, also affect the maximum speeds of rotation at which the rotators may be operated without turbulence and other objectionable effects, such as cavitation, vapor binding, and the like. It is, therefore, not possible to set forth exact rotational speed ranges for the rotators. But, the speeds of rotation will always be lower and will usually be substantially lower than those of other centrifugal pumps, wherein turbulence often occurs as the impellers thrust the fluid radially outwardly against the periphery of the pumping chamber.

Referring now to FIG. 33, the pump 14 is shown in greater detail. The pump 14 is driven by a drive shaft 30 which may be flexible or a rigid shaft depending on the distance to the drive unit. In some embodiments, the drive unit may be coupled directly to the shaft and be within the sterile field. In other embodiments, the drive unit may be maintained outside of the sterile field and coupled to the pump using a flexible drive shaft suited for use within the sterile field. The drive shaft 30 is driven as described herein by a motor (not shown). The drive shaft 30 may be driven as described in the configurations of FIGS. 38A, 38B and 39. In use for delivery of fat, the pump 14 is positioned so that the intake end 32 is coupled to a suitable harvesting canister and the discharge end 34 of the pump 14 is coupled to a delivery cannula. In other aspects, the pitch and number of spirals may vary and in alternative designs, a worm gear or screw is positioned within housing 36 in place of impeller 38.

In the illustrative embodiment of FIG. 33, the pump 14 comprises a substantially cylindrical elongate housing 36. The intake end 32 of the housing 36 is adapted so that it may be easily connected to the fat canister (see FIGS. 8-9C). A spiral-shaped foil impeller 38 is positioned within the housing 36. The impeller 38 is connected to the drive shaft 30. The drive shaft 30 is centrally positioned within the discharge end 34 of the housing 36 by a shaft stabilizer 40.

The impeller 38 functions in much the same fashion as an airfoil moving through a liquid medium. Blasius' first equation of fluid forces on a body in motion describes forces on a submerged body. The forces may be resolved into components in directions perpendicular to the motion (Y-axis) and parallel to the motion (X-axis). These forces are known as “lift” and “drag” respectively. In fluid mechanics, lift and drag are equal to the component of thrust. Rotation of the helical-shaped foil impeller 38 within the cylindrical housing 36 produces characteristics similar to the lift and drag forces produced by an airflow. That is, both high and low pressure forces are produced on either side of the rotating spiral or helical impeller 38 within the cylinder 36 which produces a thrust force to energize fluid motion within the cylinder 36.

Referring now to FIG. 36, impeller 38 is shown in a two dimensional plane. It will be observed that the profile of the impeller 38 is similar to that of an airfoil and includes a forward or leading end 42 and a trailing end 44. The impeller 38 tapers from the leading end 42 to the trailing end 44. As the impeller 38 is rotated, high and low pressure forces are created on either side of the rotating impeller. The high pressure side is defined by the surface 46 and the low pressure side is defined by the surface 48. The cord line 49 defines the angle of attack of the impeller 38 which is optimized to provide the desired fat delivery rate. In other configurations, the impeller 38 may include a screw or worm drive or multiple spiral frames including partial spiral sections driven along a common shaft adapted to pump fat.

Referring again to FIG. 33, it will be observed that the impeller 38 is wrapped or twisted to form a helical profile. The forward end of the impeller 38 as best shown in FIG. 35 presents a leading edge 50 which extends across the housing 36. From the leading edge 50, the impeller 38 defines a continuous contour to the trailing end 44 as shown in FIG. 34. The contour defines a continuous rotating passage for transforming the fat flow from a simple mass displacement at the inlet 32 to transformational flow producing a thrust and a streamline shape at the discharge end 34 of the pump 14. The transformational flow may be calculated and graphically described utilizing the Joukowsky transformation. Thus, the Joukowsky transformation may be used to calculate the thrust generated by the rotational motion of the impeller 38 within the housing 36. The rotational motion of the impeller 38 creates a thrust force which draws fat into the cylindrical housing 36 and discharges the fat through apertures extending through the shaft stabilizer 40 and through a port in the discharge end 34. The trailing end 44 of the impeller 38 is enlarged slightly at the central portion thereof for connection to the drive shaft 30. The enlarged portion 39 however tapers outwardly to the trailing end 44 of the impeller 38 such that it does not interfere with the fat flow to the discharge end 34 of the housing 36.

The helical-shaped foil profile of the impeller 38 is designed to maximize flow through the housing 36 while minimizing the potential damage to fat and cells. The impeller 38 rotation rate and the dimensions of the impeller and the housing may be adapted to provide the desired range of controllable high volume fat pumping as described herein. Turbulence however is minimized by the continuous contour of the flow passage defined by the impeller 38. The thrust generated by the high pressure side of the impeller 38 draws the fat through the pump 14 while minimizing the turbulence in the flow. The impeller 38 cooperates with the cylindrical housing 36 to form a continuous, smooth, rotating passage to transform the fat flow from a simple mass displacement at the inlet end 32 of the pump 14 to a transformational flow at the trailing end 44 of the impeller 38. In this manner, trauma to the fat and cells is minimized, yet sufficient flow is developed to shorten procedure times and provide the other benefits described above.

In various alternative embodiments, any of the centrifugal pumps described herein may be adapted to operate in a drill pump configuration. In these alternative configurations, the rotor, impeller or drive shaft is configured for engagement with a surgical drill or other controlled rotation device, coupled using a female, male, quick connect fitting or suitable flex drive connection. The fitting couples the rotational energy of the drill to the pumping components. The engagement may be male pump/female drill as in FIG. 39 or female pump and male drill drive shaft as in FIGS. 38A and 38B. In use, operating characteristics of the drill/pump combination may be provided to indicate to a user how much fat volume is being delivered by a particular drill rpm setting for a particular centrifugal pump configuration. Additionally the pump may be characterized as in FIG. 45 or user experience will inform the pump speeds useful during the clinical situation of a particular procedure.

One alternative configuration of a drill pump is provided in FIGS. 38A and 38B where a base is used to mount and secure the pump and the drill. FIG. 38A is a view of the unassembled components of the pump chamber, the base and the drill. The base has a receptacle (in phantom) sized to receive and secure the drill with a distal end of the drive shaft extending through the base as best seen in FIG. 38B. In this configuration, the drill has a male end drive shaft and the pump chamber has a female socket coupled to the rotor or other pump component depending upon configuration. FIG. 38B is components of FIG. 38A assembled for use to perform a fat transfer procedure as described herein. Suitable tubing is connected to the pump chamber inlet and outlet. As with any of the pump embodiments, a suitable priming solution may be added to the pump chamber and lines as needed to meet any pump priming needs.

FIG. 39 is a view of a centrifugal pump embodiment with a shaft extending from the pump housing. The pump shaft is sufficiently long to engage with a surgical drill. It is to be appreciated that the pump shaft extends beyond the pump casing a sufficient length and terminates with an appropriate fitting to be engaged with a receiver or collet on the operating end of the drill.

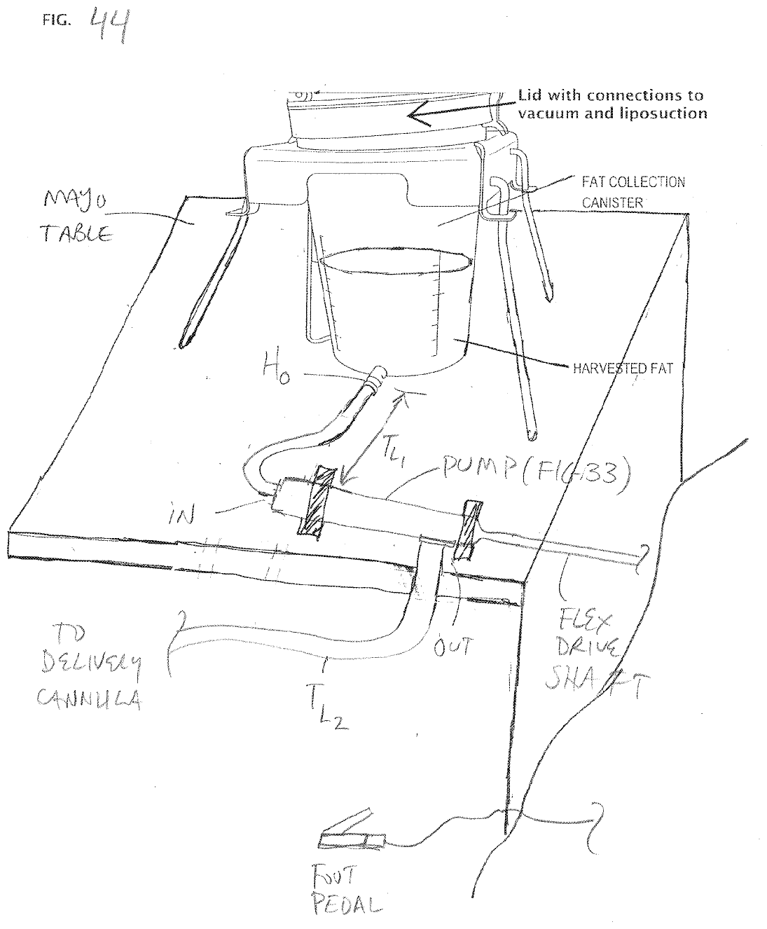

FIG. 40 is a top down view of a stabilizing plate used as a base for a centrifugal pump coupled to a surgical drill.

FIG. 41A is an interior view of a centrifugal pump with the cover removed to show the impeller and vanes. FIG. 41B is a rear view of the centrifugal pump in FIG. 41A. The drive shaft of the pump extends from the casing.

FIG. 42 is a perspective view of one arrangement of a centrifugal pump coupled to a surgical drill and configured for pumping the contents of the fat in a harvesting canister to the delivery tubing and to a delivery cannula. The centrifugal pump and surgical drill are mounted onto a plate and positioned on the tabletop within the sterile field. One or both of the centrifugal pump and the surgical drill or a portion of each of the centrifugal pump and the surgical drill are beneath the fat harvesting canister or within the footprint of the canister stand.

FIG. 43 is a perspective view of one arrangement of a centrifugal pump coupled to a drive shaft and configured for pumping the contents of the fat in a harvesting canister to the delivery tubing and to a delivery cannula. The centrifugal pump and drive shaft are mounted onto a plate and positioned on the tabletop within the sterile field. One or both of the centrifugal pump and the drive shaft or a portion of each of the centrifugal pump and the drive shaft are beneath the fat harvesting canister or within the footprint of the canister stand. A controller for adjusting the speed of the drive shaft and the pumping speed of the centrifugal pump is located outside of the sterile field. A foot pedal coupled to the drive shaft speed controller is located within the surgical field.

FIG. 44 is a perspective view of one arrangement of a centrifugal pump coupled to a drive shaft and configured for pumping the contents of the fat in a harvesting canister to the delivery tubing and to a delivery cannula. The centrifugal pump and drive shaft are mounted onto a plate and positioned on the tabletop within the sterile field. One or both of the centrifugal pump and the drive shaft or a portion of each of the centrifugal pump and the drive shaft are beneath the fat harvesting canister or within the footprint of the canister stand. A controller (not shown) for adjusting the speed of the drive shaft and the pumping speed of the centrifugal pump is located outside of the sterile field. A foot pedal coupled to the drive shaft speed controller is located within the surgical field.

FIG. 46 is a side view of one arrangement of first location (L1) and a second location (L2) for positioning a centrifugal pump and a drive unit on a tray or table. The first and second locations are selected to place all or a portion of one or both of the centrifugal pump and drive unit under the harvesting canister or within the foot print of the canister frame. The harvesting frame may be modified to increase the height of the bottom of the canister from the tope of the tray for this purpose.

It is to be appreciated that the above described centrifugal pumps may vary in the different ways by which fat may be pumped without harm or to meet the desirous pumping environment as described above. As a result, in some embodiments, a variation in the spacing between interior pump chamber components may be adopted to accommodate flow properties specific to harvested fat. In some specific embodiments, the interior chamber provides a spacing or gap in the pumping path of less than 1 mm, less than 2 mm or less than 3 mm to enable damage free fat pumping. In still other aspects, the gap or spacing is adjusted to provide spacing between interior pump components of 0.25 mm, 0.5 mm, or 0.75 mm. The above provided representative gaps for spacing within a specific pump design are provided in acknowledgement of the potential of harm to fat as it moves along a pumping pathway. As such these exemplary dimensions represent modifications to existing or new pump designs based on the principals herein. As such, embodiments of centrifugal fat pumps may provide said gap or spacing between interior surfaces of the pumping chamber, between a rotor or impeller surface and an interior of a pump casing, between a vane upper surface and the chamber interior surface or other gap within the housing where an increased spacing in the pump design would result in reduced strain, stress or otherwise enable damage free handling and pumping of the harvested fat.

As a result of the various different centrifugal pumps enabled by the present invention, the different pumping characteristics of the assorted centrifugal pump designs may be further clarified to the benefit of users. Still further, manufacturers of surgical drills or other motor control systems may wish to adapt their designs for use with one or more of the centrifugal pumps described herein. As a result, manufacturers, pump designers and users would benefit from greater characterization of fat properties and have been used previously. FIG. 45 is a table of exemplary centrifugal pump characterization chart for centrifugal pump operations in conjunction with existing surgical drills. The table relates the speed (rpm) setting for the drill model for an estimated flow rate of delivered fat. The table also includes a range of fluid percentages (FL %) for the harvested fat and for correlating the various fluid percentages to the flow rates. The table also includes representative High (H), Medium (M) and Low (L) fat flow rates. Characterization studies of fat composition and fluid amounts may be performed and recorded in the chart portions (a) and (b). A user may select different H, M and L flow rates. Drill manufacturers may provide this information as an operational suggestion as the surgical drill manufacturer tests or certifies a particular drill for use with a centrifugal fat pump.

### Additional Exemplary Uses

Any of the above described centrifugal pumps may be operated using steps similar to the basic steps outlined in FIG. 5 method 500 to accomplish a wide array of fat grafting procedures. Consider these illustrative details for exemplary cases for performing centrifugal pump enabled fat grafting procedures:

a) Reconstructive Breast—Partial Defects

For smaller volume partial defects or contour deformities: a finer cannula is used for harvesting fat, ranging in diameter of 3-4 mm. In some instances or for a portion of the harvesting a larger cannula up to 5 mm if the overall volume will be larger than 300 ml to 400 ml. In these cases the standard technique described above is used. If defects at the graft site are not due to specific scarring or issues of retraction as in radiation, then standard continuous pumping grafting introduction can be done while adjusting pumping speed or slowing or stopping the pump for grafting site assessment or manipulation. If the graft site defects are small, finer grafting cannulas sized between 14-16 gauge provide smoother transition contour. Additionally, flow rates can be commenced by selecting pump designs for lower flow rates or by selecting pump speeds configured for at lower fat delivery rates such as 25 or 50 ml/min, and increased as needed, because smaller increments are needed to achieve a visibly noticeable change in small defect sites.

b) Radiated Tissue

First, the condition of the graft bed is assessed. Often radiated patients have more significant scarring, with adherence between the dermis and underlying structures that will impede grafting. Next, regions of adherence or scarring are released as needed to allow space for grafted fat. In one aspect, a V type dissector is advanced into the graft site and used to both make space and release skin for expansion to accommodate grafted fat. In these cases one does well to keep fat finer with harvest and with introduction, by the use of 3 mm for harvest and 12 gauge for introduction. In some embodiments, a patient receiving a continuous fat grafting procedure as described herein is followed using one or more auxiliary techniques such as external expansion both pre-op and post op to optimize graft success. Also fat is uniformly spread out through multiple trajectories, and if needed into muscle as well as subcutaneous planes (see R. Khouri and BRAVA pump or other similar devices).

c) Implant Volume Inadequacy/Higher BMI Patients

In the case of patient with higher BMIs whose native breast size and proportional goals may far exceed available implant volumes, fat grafting can be a much lower risk solution than autogenous tissue and more complex flap surgery with concomitant increase surgical risk and recovery times. For these larger volume grafts in otherwise healthy and not radiated tissue, larger cannulas may be used for harvest at 4 or 5 mm. Also larger introduction cannulas as well up as is in 10 to 12 gauge. More often seen in large patients with either overzealous or aggressive mastectomies are upper pole defects, or ‘infraclavicular stepoff’ as described by Kanchwala et al. with sharp transition contours between the ‘breast versus subcutaneous adipose layers’ due to the significantly larger layer of subcutaneous adipose tissue in the higher BMI patient. In these cases the pectoralis muscle can be utilized as a well vascularized bed for optimum grafting success and one or more steps of the continuous pumping grafting method is performed to graft fat into this region.

2) Cosmetic:

a) Large volume—Buttock Augmentation

One additional benefit of the inventive methods is efficiency of transferring large volumes of fat cleanly, with the least amount of waste of both fat and time. Larger cannulas 5 mm for harvesting and introduction with large gauge microcannula selected in the range of 10-12 gauge. One or more steps of the grafting method are adapted to provide a supra-gluteal approach while avoiding deep muscular layers so as to prevent accidental intra-vascular fat injection.

b) Small Volume—Facial Rejuvenation

Much smaller volumes, in the range of 20-60 cc or in some embodiments in a range as small as 2-50 cc, harvest with smaller 2 mm cannula, inject with fat injection using 14-16 gauge blunt tipped microcannulas. Pump speeds and pump designs may be provided for operational characteristics having very low flow rates (10-25 ml/min) ideally suited to these more finely detailed cases. The pump characteristics may be adapted to deliver fat with much more precision than is available using conventional techniques and systems. Moreover, use of embodiments of the closed loop centrifugal fat pumping system and methods herein provides consistent and controlled flow rates which aid in preventing accidental over filling of the target graft site.

There are some additional mechanical advantages of a centrifugal (CF) pump as compared to a roller pump. First, CF pumps can be very simple, small designs which can be incorporated directly into the circuit within the sterile field. This allows for significant reduction in tubing length required (see for example first and second tubing lengths that total less than 4 feet combined in some embodiments). For example, in contrast, to run the tubing off the field to a pass through the roller pump and back to patient may require at least four and up to 6-8 feet of tubing. Whereas with a drill pump on the field the length of tubing could be shortened to as little as 18 inches or less (see FIGS. 38A-42 for example). Since shear stress to the graft material depends partly on length of tubing, significant decreases in length of circuit tubing, will equal less shear and less damage to adipocytes.

Second, CF pumps by their nature, provide consistent pressure, based on a given rotational speed. If upstream blockages to flow occur on roller pumps, pressure continues to build, until release, at which point all material in the tubing either explodes through a connector, or bursts through the delivery device at significantly higher pressure and accelerated rates, causing unwanted over filling of fat in a given graft area. Therefore CF pumps are safer and protect from inadvertent blow outs in the area to be grafted.

Third, power to the drill pump can be supplied by standard variable speed drills, which are readily available in even the most basic operating rooms. Being sterilizable, they are readily available to facilitate the shorter circuit to the patient on the field by simple connection to the drive shaft of the pump. In such embodiments, no high capital investment is needed to set up such a system. CF pumps are the next generation in efficiency, safety and gentle delivery of adipocyte graft material.

Fourth, CF pumps, as evidenced from decades of research in cardiovascular surgery, are also by nature gentler on cellular elements of blood as a bodily fluid. Therefore optimal flow rates, fin and impeller design can be engineered to also optimize adipocyte viability with more finesse and specificity than a simple roller pump compression design system (See FIG. 45).

Still further, as will be appreciated by considering the details of the various embodiments described herein, the inventive closed loop, continuous pumping fat grafting system provides a number of advantages over conventional syringe based or small volume transfer systems. Conventional manual systems require additional handling steps to load syringes or other equipment while the inventive system pumps directly from the harvested fat storage container to the grafting site using a single tube. The inventive pumping system provides for single hand operation without tiresome mechanical actuation required by syringe delivery. Advantageously, the inventive closed loop continuous pumping system provides real time response via foot pedal or flow control valve or clip in order to increase, decrease, pause or resume pumping action in response to real time user assessment including visual observations, measurements, tactile responses or manipulations of the grafting site, depending upon procedure. Advantageously, the inventive system reduces the need for equipment by employing the same size tubing and infusion pump for the initial tumescent infiltration step as for the later fat deliver step. In one aspect, the operating parameters of the pump have preset limits or ranges of operation for use during infusion and another set of preset limits or ranges of operation for use during fat delivery. It is to be appreciated that selection of roller and tubing materials, adjustments to roller speed, tubing properties and roller-tubing hardness may be optimized to minimize or reduce shear or other forces imparted to pumped fat while still providing sufficient or desired tumescent infusion properties or pumping characteristics. As a result, aspects of the inventive closed loop fat transfer system may use the same pump with different operating characteristics for both the infusion step as well as for the fat delivery step thereby further increasing the efficiency of fat harvesting and transfer operations.

Additional alternative pump configurations are possible based on the design factors described herein. Tuning factors of the pump interior components such as spacing and shape of impellors and rotor speeds and other design and operating conditions may be adapted based on measured or desired characteristics of the harvested fat. The properties of fat depend upon a number of variables from pre-harvesting treatments, to the cannula size used for harvesting as well as processing techniques used within the harvesting canister or canister type (FIGS. 8-9C). It is to be appreciated that pre-treatment, harvesting and handling of fat may be standardized and characterized in order to better match fat characteristics and qualities with centrifugal pump designs suited to high volume pumping rates while maintaining fat integrity. (see FIG. 45)

Additional details related to high volume fat graphing operations and long term viability of high volume grafts may be appreciated by reference to International Patent Application No. PCT/US2017/029952, titled “A CLOSED LOOP SYSTEM FOR DIRECT HARVEST AND TRANSFER FOR HIGH VOLUME FAT GRAFTING,” filed Apr. 27, 2017; U.S. patent application Ser. No. 15/139,767, titled, “A SIMPLE CLOSED LOOP SYSTEM FOR DIRECT HARVEST AND TRANSFER FOR HIGH VOLUME FAT GRAFTING” filed on Apr. 27, 2016; and to U.S. Provisional Patent Application No. 62/426,159, titled “CLOSED LOOP SYSTEMS FOR DIRECT HARVEST AND TRANSFER FOR HIGH VOLUME FAT GRAFTING,” filed Nov. 23, 2016, each of which is herein incorporated by reference in its entirety.

Additional details are provided in U.S. Patent Application Publication No. US2015/0209565, U.S. Patent Application Publication No. US2005/0084961, U.S. Pat. No. 8,632,498, U.S. Patent Application Publication No. US2009/0287190, U.S. Patent Publication No. 2013/0324966 and U.S. Patent Application Publication No. 2012/0253317, as well as in these references:
1. Spear S L, Wilson H B, Lockwood M D. Fat injection to correct contour deformities in the reconstructed breast. Plast Reconstr Surg. 2005; 116:1300-1305.
2. Coleman S R, Saboeiro A P. Fat grafting to the breast revisited: Safety and efficacy. Plast Reconstr Surg. 2007; 119:775-785;
3. Delay E, Garson S, Tousson G, Sinna R. Fat injection to the breast: Technique, results, and indications based on 880 procedures over 10 years. Aesthet Surg J. 2009; 29:360-376.
4. Khouri R K, Rigotti G, Cardoso E, Khouri R K Jr, Biggs T M. Megavolume autologous fat transfer: Part I: Theory and Principles. Plast Reconstr Surg. 2014; 133:550-557.
5. Khouri, Roger K. M.D.; Rigotti, Gino M.D.; Cardoso, Eufemiano M.D.; Khouri, Roger K. Jr. B.S.; Biggs, Thomas M. M.D. Megavolume Autologous Fat Transfer: Part II. Practice and Techniques. Plast Reconstr Surg. 2014; 133:1369-1377.
6. Khouri, Roger K., Jr., B S, Khouri, Raoul-Emil R., Lujan-Hernandez, Jorge R., et al, “Diffusion and Perfusion: The Keys to Fat Grafting,” Plast Reconstr Surg Glob Open. 2014 September; 2(9):e220, published online 2014 Oct. 7.
7. Sinno S, Wilson S, Brownstone N, Levine S M. Current Thoughts on Fat Grafting: Using the Evidence to Determine Fact or Fiction. PRS 2016 March; 137(3):818-24.
8. Kanchwala S K, Glatt B S, Conant E F, Bucky L P. Autologous fat grafting to the reconstructed breast: The management of acquired contour deformities. Plast Reconstr Surg. 2009; 124:409-418.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of performing a fat grafting process on a patient, comprising:
harvesting an amount of fat from a donor site of the patient; and
operating a centrifugal pump for delivering a portion of the amount of fat into a grafting site of the patient at a rate of between 100 ml/min to 150 ml/min.

2. The method of claim 1 further comprising:
operating the centrifugal pump for delivering a second portion of the amount of fat into a second grafting site of the patient at a rate of between 100 ml/min to 150 ml/min.

3. The method of claim 1 wherein the amount of fat from the harvesting step is retained in a storage container and the delivering step is performed by pumping the fat from the storage container directly to the grafting site.

4. The method of claim 3 wherein the fat from the harvesting step and delivered by operating a centrifugal pump remains within the sterile field established for performing the fat grafting process.

5. The method of any of the above claims wherein the portion of the amount of fat is 700 cc and the delivering step is less than 22 minutes.

6. The method of any of claims 1-5 wherein the grafting site and the second grafting site are on a breast.

7. The method of any of claims 1-5 wherein the grafting site is on a right breast and the second grafting site is on a left breast.

8. The method of claims 1-7 wherein the harvesting an amount is between about 1-5 ml, 1-50 ml, 1-100 ml, 100-200 ml, 100-500 ml, 100-1000 ml or 100-2000 ml of harvested fat.

9. The method of claims 1-8 wherein fat grafting process is performed as part of a breast reconstruction procedure or a cosmetic breast revision procedure.

10. The method of claims 1-8 wherein fat grafting process is performed as part of a cosmetic body contouring procedure.

11. The method of claim 9 or 10 further comprising adapting a portion of the harvesting step or the operating step to accommodate for a partial defect at the grafting site.

12. The method of claim 9 or 10 further comprising adapting a portion of the harvesting step or the operating step to accommodate for a scarring from radiated tissue at the grafting site.

13. The method of claim 9 or 10 further comprising adapting a portion of the harvesting step or the operating step to accommodate for an implant volume inadequacy at the grafting site.

14. The method of any of claims 1-13 wherein the operating step is performed by imparting rotation of the centrifugal pump via a magnetic coupling.

15. The method of any of claims 1-14 wherein the operating step is performed by imparting rotation of the centrifugal pump using a surgical drill, or a motor drive or a flexible cable drive.

16. The method of any of claims 1-15 wherein the amount of fat delivered is measured by a component of the centrifugal pump or a flow meter coupled to a centrifugal pump outlet or by operating the centrifugal pump at a predetermined speed setting for a predetermined time duration.

17. A method of performing a fat grafting process on a patient, comprising:
harvesting an amount of fat from a donor site of the patient into a storage container;
positioning a delivery cannula into a first portion of a grafting site of the patient;
adjusting the speed of a centrifugal pump to pump a portion of the amount of fat directly from the storage container into the first portion of the grafting site of the patient at a rate of between 10 ml/min to 350 ml/min until a first volume of fat is delivered;
positioning a delivery cannula into a second portion of a grafting site of the patient; and
adjusting the speed of the centrifugal pump to pump a second portion of the amount of fat directly from the storage container into the second portion of the grafting site of the patient at a rate of between 10 ml/min to 350 ml/min until a second volume of fat is delivered, wherein the centrifugal pump remains within the sterile field and in fluid communication with the storage container.

18. A method of performing a fat grafting process on a patient, comprising:
harvesting an amount of fat from a donor site of the patient into a collection canister; positioning a delivery cannula into a first portion of a grafting site of the patient;
flowing a portion of the amount of fat directly from the collection canister through a first tube and into an inlet of a centrifugal pump;
adjusting the speed of the centrifugal pump to deliver fat from a collection canister through a pump outlet into a second tubing connected to the delivery cannula into the first portion of the grafting site of the patient at a rate of between 10 ml/min to 350 ml/min until a first volume of fat is delivered; and
positioning the delivery cannula into a second portion of a grafting site of the patient; and
flowing a second portion of the amount of fat directly from the collection canister through the delivery cannula into the second portion of the grafting site of the patient by adjusting the speed of the centrifugal pump to deliver fat at a rate of between 10 ml/min to 350 ml/min until a second volume of fat is delivered, wherein the first tube and the second tube remain completely within the sterile field and the combined length of the first tube and the second tube is less than four feet.

19. The method of claim 17 or 18 further comprising operating the centrifugal pump used in the flowing a portion steps continuously while performing the positioning the delivery cannula steps.

20. The method of claim 17 or 18 further comprising operating the centrifugal pump used in the flowing a portion step at a first flow rate during the pumping a portion step and at a second different flow rate during the flowing a second portion step.

21. A method of performing a fat grafting process on a patient, comprising:
harvesting an amount of fat from a donor site of the patient into a storage container; positioning a delivery cannula into a first port of a grafting site of the patient;
operating a centrifugal pump at a first speed to pump a portion of the amount of fat directly from the storage container into the first port of the grafting site of the patient until a first volume of fat is delivered;
positioning the delivery cannula into a second port of a grafting site of the patient; and
operating the centrifugal pump at a second speed to pump a second portion of the amount of fat directly from the storage container into the second port of the grafting site of the patient until a second volume of fat is delivered, wherein the centrifugal pump used to pump the fat is at least partially underneath or directly adjacent to the storage container such that a tube connecting a storage container outlet to a pump inlet is less than 12 inches long.

22. The method of performing a fat grafting process on a patient of claim 21 wherein one or both of the steps of operating a centrifugal pump is performed at a rate of 100 ml/min to 350 ml/min.

23. The method of performing a fat grafting process on a patient of claim 21 wherein the grafting site of the patient comprises a portion of the patient's face.

24. The method of performing a fat grafting process on a patient of claim 21 wherein the grafting site of the patient comprises a portion of the patient's buttocks.

25. The method of performing a fat grafting process on a patient of claim 21 wherein the grafting site of the patient comprises a portion of the patient's breast.

26. The method of performing a fat grafting process on a patient of claim 21 wherein the steps are performed to accomplish a cosmetic effect at the grafting site.

27. The method of performing a fat grafting process on a patient of claim 21 wherein the steps are performed to accomplish a reconstructive effect at the grafting site.

28. The method of performing a fat grafting process on a patient of claim 21 wherein the steps are performed to accomplish a structural revision effect at the grafting site.

29. The method of performing a fat grafting process on a patient of claim 21 wherein the steps are performed to accomplish a bilateral breast construction at the grafting site.

30. The method of performing a fat grafting process on a patient of claim 21 wherein the steps are performed as part of a cosmetic body contouring procedure.

31. The methods of any of claims 1-30 further comprising manipulating or treating at least a portion of the amount of fat from a harvesting step.

32. The method of claim 31 wherein a step of manipulating or treating comprises one or more of a step of centrifuging harvested fat;
a step of processing fat to meet a characterization property of FIG. 45;
a step of filtering harvested fat;
a step of gravitational separation of harvested fat;
or a step of mixing harvested fat;
or a step of incorporating one or more substances into harvested fat helpful to ensuring viability of fat cells for the pumping process or for survival after a grafting process.

33. The method of any of claims 1-32 further comprising positioning the collection canister and the centrifugal pump in a position adjacent the patient the patient's fat may be harvested and the fat delivered using the centrifugal pump without using any tubing longer than three feet.

34. A method of performing a closed loop fat harvesting and fat grafting process on a patient, comprising:
(1) harvesting an amount of fat from a donor site of the patient; and (2) delivering a portion of the amount of harvested fat into a grafting site of the patient by adjusting the speed of a centrifugal pump to deliver the harvested fat at a rate of between 10 ml/min to 350 ml/min.

35. The method of claim 34 further comprising: delivering a second portion of the amount of fat into a second grafting site of the patient at a rate of between 10 ml/min to 150 ml/min by adjusting the speed of the same centrifugal pump.

36. The method of claim 34 wherein the amount of fat from the harvesting step can be retained in a storage container and the delivering step can be performed by pumping the fat from the storage container directly to the grafting site using the centrifugal pump.

37. The method of claims 34 to 36 wherein the centrifugal pump is operated so that a portion of the amount of fat is 700 cc and the delivering step is less than 22 minutes.

38. The method of any of claims 34-37 wherein the grafting site and the second grafting site can be on a breast.

39. The method of any of claims 34-37 wherein the grafting site can be on a right breast and the second grafting site can be on a left breast.

40. The method of any of claims 34-39 wherein the delivery of an amount of harvested fat to a grafting site with a centrifugal pump speed controlled to deliver an amount of harvested fat is from about 1-5 ml, 1-50 ml, 1-100 ml, 100-200 ml, 100-500 ml, 100-1000 ml or 100-2000 ml.

41. The method of claim 40 wherein the harvested fat is delivered by operation of the centrifugal pump connected directly to the fat harvesting container.

42. The method of any of claims 34-40 wherein the speed of the centrifugal pump is selected to deliver harvested fat at a rate for a fat grafting process performed as part of a breast reconstruction procedure or a cosmetic breast revision procedure, or for a fat grafting process performed as part of a cosmetic body contouring procedure, or at a rate adapted as a result of a portion of the harvesting step or the delivering step adjusted to accommodate for a partial defect at the grafting site or for adapting a portion of the harvesting step or the centrifugal pump operation during the delivering step to accommodate for a scarring from radiated tissue at the grafting site or for adapting a portion of the harvesting step or the delivering step to accommodate for an implant volume inadequacy at the grafting site.

43. The method of any of claims 34-42 the method further comprising one or more or a combination of the steps of performing a fat grafting process on a patient including: (1) harvesting an amount of fat from a donor site of the patient into a storage container; (2) treatment or preparation of the fat by centrifuging, filtering, or gravitational separation; (3) positioning a delivery cannula into a first portion of a grafting site of the patient; (4) pumping a portion of the amount of fat directly from the storage container via a centrifugal pump into the first portion of the grafting site of the patient at a rate of between 100 ml/min to 350 ml/min until a first volume of fat is indicated on a pump flow meter or other measure used during the pumping step; and (5) positioning a delivery cannula into a second portion of a grafting site of the patient; and (6) pumping a second portion of the amount of fat directly from the storage container via the same centrifugal pump into the second portion of the grafting site of the patient at a rate of between 10 ml/min to 350 ml/min until a second volume of fat is indicated on a pump flow meter or other measure used during the pumping step, wherein the centrifugal pump used during the pumping step is in continuous communication with the storage container during the positioning a delivery cannula steps.

44. A system for closed loop fat harvesting and continuous fat pumping, comprising:
a centrifugal pump having an inlet, an outlet and a drive shaft;
a first length of tubing for connecting an outlet of a collection canister to the inlet;
a second length of tubing for connecting the outlet to a delivery cannula; and
a drive system coupled to the drive shaft wherein operation of the drive system rotates the drive shaft to pump fat from the collection canister to the delivery cannula at a rate of fat delivery.

45. The system of claim 44 wherein the first length of tubing is selected to extend from the outlet of the collection canister to the inlet when the centrifugal pump is at least partially underneath or adjacent or in proximity to the collection canister.

46. The system of claim 44 wherein the delivery cannula is a 12 gauge microinjection cannula, a 14 gauge microinjection cannula or a 16 gauge microinjection cannula.

47. The system of claim 44 wherein a fat harvesting cannula used to fill the collection canister is a 2 mm size cannula, a 3 mm size cannula, a 4 mm size cannula or a 5 mm size cannula.

48. The system of claim 44 wherein the first length of tubing is less than 12 inches long.

49. The system of claim 44 wherein the second length of tubing is less than 48 inches long.

50. The system of claim 44 wherein the centrifugal pump has operating characteristics in the fat delivery mode adapted to perform according to a pump characterization table as in FIG. 45.

51. The system of claim 44 wherein the centrifugal pump has an impeller without vanes, an impeller with vanes or an impeller shaped as a spiral drive, a helical drive, a worm drive of a section of any of the above.

52. The system of any of claim 44-51 wherein the storage canister has a size to accommodate the volumes of claim 7.

53. The system of any of claim 44-51 wherein the operation of the drive system is adapted and configured to rotate the drive shaft of the centrifugal pump to provide fat as part of any surgical procedure as in claims 1-7, 9-13 or to provide fat pumping rates as in claims 1, 2, 5, 14-22 or to provide fat pumping rates as provided in a pump-fat characterization table as in FIG. 45.

54. The system of claims 41-53 wherein the drive shaft is any of a surgical drill, a motor drive, a magnetic drive or a flexible couple drive.

55. A pump, comprising:
a housing with an interior chamber;
an inlet in communication with the interior chamber;
an outlet in communication with the interior chamber;
an impeller within the interior chamber and shaft coupled to the impeller wherein rotation of the impeller shaft causes a volume of harvested fat at the inlet to move through the interior chamber with the impeller and exit of the interior chamber via the outlet.

56. The pump of claim 55 wherein the impeller is rotated by a magnetic drive coupled to the housing.

57. The pump of claim 55 wherein the impeller shaft extends outside of the pump housing via a aperture in the housing.

58. The pump of claim 57 wherein the impeller is rotated by coupling a portion of the impeller shaft outside of the pump housing to a drill.

59. The pump of claim 57 wherein the impeller is rotated by coupling a portion of the impeller shaft outside of the pump housing to a motor.

60. The pump of claim 57 wherein the impeller is rotated by coupling a portion of the impeller shaft to a flexible driver system.

61. The pump of claim 57 wherein the impeller shaft is a flexible cable.

62. The pump of claim 55 wherein the impeller shaft has a fitting to receive a drive shaft via an aperture in the housing adjacent to the fitting.

63. The pump of claim 62 wherein the fitting and the drive shaft are keyed to have a complimentary, releaseable engagement.

64. The pump of claim 62, wherein the releaseable engagement is male/female, socket/driver, start shaped, indexed or other complementary engagement pattern adapted and configured for coupling the fitting and the drive shaft.

65. The pump of claim 55 wherein a speed of rotation of the impeller provides a fat flow rate of between 10-50 ml/min.

66. The pump of claim 55 wherein a speed of rotation of the impeller provides a fat flow rate of between 75-200 ml/min.

67. The pump of claim 55 wherein a speed of rotation of the impeller provides a fat flow rate of between 200-400 ml/min.

68. The pump of claim 55 wherein a first speed of impeller rotation provides a user determined low volume fat pumping rate.

69. The pump of claim 55 wherein a second speed of impeller rotation provides a user determines medium volume fat pumping rate.

70. The pump of claim 55 wherein a third speed of impeller rotation provides a user determined high volume fat pumping rate.

71. The pump of claim 55 further comprising a drive system for controlling impeller rotation speed wherein the drive system if adapted and configured to provide adjustable impeller speed or preselected impeller speed as in any of claim 65-70 or within the range of 10 ml/min to 400 ml/min.

72. The pump of any of claims 55-71 wherein the pump, the fat and the impeller rotation speed are characterized and provided as in FIG. 45.

73. The pump of any of claims 55-72 wherein the impeller has a smooth pumping surface as in any of FIGS. 18-32.

74. The pump of any of claim 55-72 wherein the impeller has one or more raised surfaces in a pumping surface as in any of FIG. 13-17, 38A, 38B. 39 or 41.

75. The pump of any of claims 55-72 wherein the impeller is a spiral drive, a helix drive or a worm gear drive or a section of any of these drives.
